(19) United States
(12) Patent Application Publication
(10) Pub. No.: US 2021/0361456 A1
(43) Pub. Date: Nov. 25, 2021

(54) APPARATUS AND METHOD FOR STABILIZING A HUMAN ANATOMICAL JOINT

(71) Applicant: Stoko Design, Inc., Vancouver (CA)

(72) Inventors: Nick RUTCKYJ, Vancouver (CA); Andrew CAMPBELL, Squamish (CA); Kevin REILLY, Vancouver (US)

(21) Appl. No.: 17/222,903

(22) Filed: Apr. 5, 2021

Related U.S. Application Data
(63) Continuation-in-part of application No. 16/216,668, filed on Dec. 11, 2018.
Application No. 16/216,668 is a continuation-in-part of application No. 16/677,268, filed on Nov. 7, 2019.
(60) Provisional application No. 63/005,974, filed on Apr. 6, 2020.
(60) Provisional application No. 62/599,675, filed on Dec. 15, 2017.
(60) Provisional application No. 62/640,513, filed on Mar. 8, 2018.
(60) Provisional application No. 62/758,549, filed on Nov. 10, 2018.

(51) Int. Cl. A61F 5/01

(52) U.S. Cl. CPC A61F 5/0123; A61F 2005/0155; A61F 5/013

(57) ABSTRACT

The present invention provides a wearable brace garment configured for stabilizing an articulating joint of a wearer. The garment comprises a tension member disposed along a three-dimensional spatial path about the joint in conduits on or within the garment. The tension member comprises a plurality of strands substantially inextensible along a longitudinal direction. The garment further comprises a web portion comprising the strands laterally spread, each strand disposed in a corresponding strand conduit. A tensioner is disposed on the garment and arranged for adjusting a tension in the tension member. A tension evener is disposed on the garment tension-wise in communication with the tensioner and with the strands of the tension member. The tension evener is arranged for evening tension among the plurality of inextensible strands of the tension member within the web portion. The tension evener may comprise a whippletree. In some embodiments the whippletree may be a multi-tier whippletree.

### CROSS-REFERENCE TO RELATED APPLICATIONS

The present application claims priority under 35 U.S.C. § 119(e) from U.S. Provisional Patent Applications Ser. No. 63/005,974, filed Apr. 6, 2020; and is further related to, and claims priority under 35 U.S.C. § 120 from U.S. patent application Ser. Nos. 16/216,668 and 16/677,268, Ser. No. 16/216,668 filed Dec. 11, 2018 which claims priority under 35 U.S.C. § 119(e) from U.S. Provisional Patent Applications Ser. Nos. 62/599,675 and 62/640,513; filed Dec. 15, 2017, and Mar. 8, 2018, respectively, and Ser. No. 16/677,268 filed Nov. 7, 2019 which claims priority under 35 U.S.C. § 119(e) from U.S. Provisional Patent Application Ser. No. 62/758,549, filed Nov. 10, 2018; the disclosures of which are incorporated in their entirety herein.

### BACKGROUND OF THE INVENTION

### Field of the Invention

This present invention relates to the medical field as exemplified by IPC class A61 and more particularly to apparatus and associated methods for stabilizing articulating joints of the human body, including devices adapted to facilitate walking. In one aspect, it relates to an apparatus for stabilizing the human knee and the operation of such an apparatus configured and arranged for treating damaged ligaments in the knee.

### Description of the Related Art

Orthopedic braces are used to stabilize joints between the limbs of the human anatomy in cases where the joints or the limbs articulating about them have sustained damage. Braces have been employed to stabilize knees, ankles, elbows and wrists in this way. The brace is applied to reduce strain on the injured limb or joint while permitting the limb or joint to still perform its function, thereby minimizing the risk of further damage.

Several knee brace products have been developed to more specifically protect the ligaments of the knee, including the anterior cruciate ligament (ACL), posterior cruciate ligament (PCL), and medial collateral ligament (MCL). These are the ligaments most often damaged in when knees are overstrained, particularly by individuals involved in strenuous sporting activity. The lateral collateral ligament (LCL) may also sometimes become strained. The brace products for protecting these ligaments vary greatly in technology, function, and efficacy. One such product is disclosed in U.S. Patent Publication No. 2019-0374361 A1, assigned to the assignee of the present application.

Some products are focused on providing mechanical encapsulation that still allows articulation of the joint. Such products tend to involve rather heavy hinge mechanisms and are generally bulky and not particularly aesthetic. At the other extreme there are several products that assume the form of a garment, different forms of such garments employing different technologies to produce compressive forces in the general vicinity of the knee in an attempt to stabilize it and protect the above ligaments. Some garments fall short in providing enough compression. Others provide adequate overall compression but do not direct the compression to key anatomical areas.

Knee brace products have been developed to address osteoarthritis, focusing on unloading the pressure on the medial compartment cartilage. There are two main styles of unloader knee braces: condylar pads and force straps. The condylar pad design used a three-point bending mechanism centered around an applied pressure to the condyle pad to unloading moment to the knee. The force strap design uses a strap that wraps around knee to apply the moment. In both cases, the added pressure from the condyle pad or strap can cause discomfort, restrict blood circulation. Braces that are configured to unload the medial compartment are also known to apply pressure to the peroneal nerve causing a numbing or painful situation. Studies have indicated that more than half of patients discontinue using these braces due to these issues. Unloader knee braces also suffer from many of the same issues as a traditional brace, including migration, comfort, bulk, and weight that lead to reduced compliance.

There remains a need for an unloader knee brace that accomplish a valgus moment and that does not interfere with blood circulation in the popliteal artery. Also, to avoid pain and discomfort due to prolonged use. There additionally remains a need for a brace that does not apply condyle force against the lateral condyle. There remains a need for unloader braces to address additional issues cited to reduce compliance with these devices.

### SUMMARY OF THE INVENTION

In a first aspect, a garment is presented that is arranged for enveloping at least in part an articulating joint of a human body, the garment comprising one or more tension members each disposed longitudinally along a corresponding predetermined curved three-dimensional spatial path within or on a matrix of a garment material, the corresponding predetermined paths spatially relating the one or more tension members to at least one natural ligament of the joint. The one or more tension members may each comprise one or more strands of a substantially longitudinally inextensible material; and the garment material may comprise conduits disposed within or on the garment material and arranged to receive the one or more strands of each tension member. Each conduit may a lower coefficient of friction with respect to the strands than the coefficient of friction of the garment material with respect to the strands. Each conduit may comprise one or more tube of material different from the garment material, the tubes arranged lengthwise along the predetermined three-dimensional spatial path of the corresponding tension member.

The garment may further comprise at least one tensioner disposed in line with at least one of the one or more tension members for tensioning the at least one tension member. At least one of the one or more tension members may further comprise at least one tension regulator disposed for balancing tension differences between different strands of the at least one tension member.

When the articulating joint is a knee with a patella in a leg of the human body, the path of at least one of the one or more tension members may comprise a lateral segment disposed to extend generally vertically proximate and lateral to the patella and a medial segment disposed to extend generally vertically proximate and medial to the patella. The garment may further comprise at least one portion of inextensible fabric connecting the medial segment of the at least one tension member to the lateral segment of the at least one tension member around the posterior of the leg.

The lateral and medial segments of the at least one tension member may further be disposed to cross over each other above and below the knee at respectively upper and lower crossover points. The garment may further comprise at least one of a portion of inextensible fabric connecting the medial segment of the at least one tension member to the lateral segment of the at least one tension member over the anterior of the knee above the upper crossover point and a portion of inextensible fabric connecting the medial segment of the at least one tension member to the lateral segment of the at least one tension member over the anterior of the knee below the lower crossover point.

In general, in the case of the articulating joint being a knee, the garment may comprise a portion of inextensible fabric laterally joining two segments of the tension member in tension with respect to each other horizontally over the leg.

The garment may further comprise a belt disposed for tightening the garment about a waist of the human body and a stabilizing connector connecting the at least one tension member to the belt, wherein the stabilizing connector is composed of a substantially inextensible material.

The three-dimensional spatial path of at least one tension member may extend around a limb articulating at the joint. The three-dimensional spatial path of at least one tension member may be arranged so that tension in the at least one tension member produces or exerts a compressive force proximate the joint. The at least one longitudinal portion of the one or more tension members may comprise a length of substantially inextensible fabric.

In another aspect, a garment is presented that comprises a closed tensionable loop arranged to freely move along a conduit within or on the garment, wherein the conduit is arranged to cross over itself between a lateral and a medial side on the anterior of a leg of the user at least one of above and below a knee of the leg. In some embodiments, the conduit is arranged to cross over itself between a lateral and a medial side on the anterior of a leg of the user both above and below a knee of the leg. The tensionable loop is disposed in or on the garment for applying pressure to at least one of a lateral collateral ligament and a medial collateral ligament of the user when the garment is worn by the user and the tensionable loop is tensioned. The garment may further comprise a tensioner disposed for tensioning the tensionable loop. The garment may further comprise a belt disposed to fit around the waist of the user and the tensionable loop may be anchored to the belt. The conduit may be arranged along a path that circles the leg at a distal region of a triceps surae of the leg.

The garment may further comprise an anchor member and the tension member and anchor member may jointly at least partially, or in major part, encircle the thigh. In other embodiments, the tension member at least partially, or in major part, encircles the thigh.

The tensionable loop may comprise a tension member extending along the conduit sandwiched between layers of low friction material. The garment may be fashioned from a garment material and the conduit may be contained within the garment material and be laterally demarcated by stitching. The stitching may comprise a low friction fiber. The tensionable loop may comprise a tension member extending along the conduit and the conduit may comprise a low friction tube, which may be a collated tube. The tensionable loop may comprise a tension member made of a substantially longitudinally inextensible material and the inextensible material may be flexible.

In a further aspect, a method is presented for stabilizing a knee of a human user leg, comprising: applying around a knee of the user a wearable garment comprising a garment leg, a garment knee, a tensioner and a closed tensionable loop arranged around the leg of the garment to freely move longitudinally along a conduit within or on the garment, wherein the conduit is arranged to cross over itself between a lateral and a medial side on the anterior of the garment leg above and below the knee of the garment and the tensioner is disposed and arranged for adjusting a tension the loop; and operating the tensioner to adjust a tension in the loop.

The applying may comprise: arranging the garment on the user to position a first portion of the loop against a distal region of a triceps surae of the user leg; positioning a second portion of the loop on or above a thigh of the user leg; and positioning on the medial and lateral sides of a patella of the user leg proximate two collateral ligaments of the user knee third and fourth portions of the loop located on the loop between the first and second portions. The positioning of the second portion of the loop may comprise positioning the second portion of the loop against a posterior of the thigh of the user leg.

In a further aspect, a method is provided for manufacturing a wearable garment for stabilizing a knee of a user, the method comprising: forming a garment covering at least in part at least a knee, an adjoining triceps surae, and a distal portion of an adjoining thigh; establishing at least one of in and on the garment a conduit for receiving a tensionable loop, the conduit arranged to cross over itself between a lateral and a medial side on the anterior of the garment at least one of above and below the garment knee; threading along a path through the conduit a tensionable member; establishing one of in and on the loop a tensioner for adjusting a tension the loop.

Threading the tensionable member may comprise threading a tensionable member that is substantially inextensible. Threading the tensionable member may comprise threading a tensionable member that is flexible and substantially inextensible. Threading the tensionable member may comprise threading a tensionable member made from one or more of PTFE, stainless steel, Nylon®; Kevlar®; one or more ultra-high molecular weight polyethylene-based fiber, a fiber with a diamond weave. Threading a tensionable member made from the fiber with a diamond weave may comprise threading a tensionable member made from one of cotton, polyester, polypropylene, and Technora®.

Establishing the conduit may comprise arranging the conduit to cross over itself above and below the knee. Forming the garment may comprise extending the garment to a waist of the user, forming a belt around the waist. Establishing the tensioner may comprise establishing the tensioner on the belt. Establishing the tensioner in or on the loop may comprise establishing the tensioner on a thigh of the garment.

In a further embodiment, a wearable garment is presented for stabilizing a knee of a user comprising: a closed tensionable loop arranged to freely move along a conduit disposed within or on the garment, the conduit extending along a conduit path having relative to the knee a lateral portion and a medial portion; and an adjustable tensioner in or on the loop for adjusting a tension in the loop; wherein the lateral and medial portions of the conduit path pass a patella of the user knee proximate the lateral and medial collateral ligaments of the user knee respectively and in which the lateral and medial portions of the conduit path closely approach each other at least one point, the at least one point being at least one of below and above the user knee.

The lateral and medial portions of the conduit path may closely approach each other at a first point below the user knee and at a second point above the user knee. The tensionable loop may be disposed for applying pressure to at least one of the lateral collateral ligament and a medial collateral ligament of the user when the garment is worn by the user and the tensionable loop is tensioned. The conduit may be arranged along a path that substantially circles the leg at a distal region of a triceps surae of the leg. The tensionable loop may comprise a tension member extending along the conduit. The tensioner may be disposed on the thigh of the user. The tension member may be made of a substantially longitudinally inextensible material and the material may be flexible. The conduit may comprise a low friction tube and the tube may be collated.

In a further aspect a method is presented for manufacturing a wearable garment for stabilizing a knee of a user, the method comprising: forming a garment covering at least in part at least a knee, an adjoining triceps surae, and a distal portion of an adjoining thigh; establishing at least one of in and on the garment a conduit for receiving a tensionable loop, the conduit extending along a conduit path having relative to the knee a lateral portion and a medial portion, the lateral and medial portions of the conduit path closely approaching each other at least one point, the at least one point being at least one of below and above the user knee; threading along a path through the conduit a tensionable member; establishing one of in and on the loop a tensioner for adjusting a tension the loop.

Threading the tensionable member may comprise threading a tensionable member that is substantially inextensible. Threading the tensionable member may comprise threading a tensionable member that is flexible and substantially inextensible. Threading the tensionable member may comprise threading a tensionable member made from one or more of PTFE, stainless steel, Nylon®; Kevlar®; one or more ultra-high molecular weight polyethylene-based fiber, a fiber with a diamond weave.

Threading a tensionable member made from the fiber with a diamond weave may comprise threading a tensionable member made from one of cotton, polyester, polypropylene, and Technora®. Establishing the conduit may comprise arranging the conduit to have the lateral and medial portions of the conduit path closely approaching each other below and above the user knee. Forming the garment may comprise extending the garment to a waist of the user, forming a belt around the waist, in which establishing the tensioner comprises establishing the tensioner on the belt. Establishing the tensioner in or on the loop may comprise establishing the tensioner on a thigh of the garment.

In further embodiments, a wearable brace garment is presented for stabilizing an articulating joint of a wearer, the garment arranged for enveloping at least in part the joint, the garment comprising: a tension member disposed spatial path about the joint in conduits on or within the garment, the tension member comprising a plurality of strands substantially inextensible along a longitudinal direction; a web portion comprising the strands laterally spread, each strand disposed in a corresponding strand conduit; a tensioner disposed on the garment and arranged for adjusting a tension in the tension member; a tension evener disposed on the garment tension-wise in communication with the tensioner and with the strands of the tension member, the tension evener arranged for evening tension among the plurality of inextensible strands of the tension member. The tension evener may comprise a whippletree that may be a multi-tier whippletree. The whippletree may more particularly be a two-tier or three-tier whippletree.

The whippletree may employ pulleys and/or threadable members. The threadable members may include beads, rings, eyelets, blocks, loops of inextensible fabric, loops in inextensible cables, and whipple bars for engaging with the inextensible strands of the tension member. The pulleys, threadable beads, rings, eyelets, blocks and whipple bars may be formed of any material capable of withstanding the tensions applied, including wood, metal, plastic and other suitable polymer materials. The polymer elements may be formed by injection molding or three-dimensional printing technology.

The garment may be made of a garment material and each conduit may have a lower coefficient of friction with respect to the strands than the coefficient of friction of the garment material with respect to the strands. Each of the conduits may comprise a tube of material different from the garment material. The three-dimensional spatial path of the tension member may be arranged so that tension in the tension member produces a compressive force proximate the joint. The tensioner may be engaged with the tension evener so as to provide a mechanical advantage in applying tension to the tension evener. The joint may be one of an ankle, elbow, wrist, shoulder, hip, neck, spine, and one or more vertebrae of the wearer.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a drawing showing the general structure of the human left knee with the patella and its adhesions deleted for the sake of clarity.

FIG. 2 is a drawing of a first embodiment of a bracing garment for bracing the human left knee.

FIG. 3 is a drawing of another embodiment of a bracing garment for bracing the human left knee.

FIG. 4 is a drawing of another embodiment of a bracing garment for bracing the human left knee

FIG. 5 shows the thigh portion of FIG. 2 enlarged and with more detail.

FIG. 6A shows an arrangement of a tension member and conduit in one embodiment

FIG. 6B shows an arrangement of tension members and conduits in another embodiment.

FIG. 6C shows an arrangement of a tension member and conduit in another embodiment.

FIG. 7 is a flow chart diagram of a method for stabilizing a knee of a human leg.

FIG. 8 is a flow chart diagram of a method of manufacturing a bracing garment for bracing the human knee.

FIG. 9 is a drawing of a further embodiment of a bracing garment for bracing the human left knee.

FIG. 10 is a flow chart diagram of another method of manufacturing a bracing garment for bracing the human knee.

FIG. 11 is a drawing of another embodiment of a bracing garment for bracing the human left knee.

FIG. 12 is a drawing showing an embodiment of a tension regulator for use with the tension member of FIG. 6B.

FIG. 13 is a drawing showing an embodiment of a tension regulator for use in regulating the tension at the two ends of the tension member of FIG. 11.

FIGS. 14 A and 14B are two perspective views of a brace garment with a tensioner and a tension evener.

FIG. 15 is a cut-away view of a component of the garment of FIGS. 14A and 14B showing how a plurality of strands of a compound inextensible cable may be guided by conduits in a calf grip of the garment.

FIGS. 16A to 16F are schematic views showing different embodiments of a two-tier whippletree tension evener of the garment of FIGS. 14A and 14B.

FIG. 17A to FIG. 17C are schematic views showing different embodiments of a three-tier whippletree tension evener of the garment of FIGS. 14A and 14B.

FIGS. 18A and 18B are perspective views showing an embodiment of a brace garment for supporting a shoulder joint of a wearer.

FIGS. 19A and 19B are perspective views showing another embodiment of a brace garment for supporting a shoulder joint of a wearer.

FIG. 20 shows a view of an embodiment of a brace garment for supporting an ankle joint of a wearer.

FIGS. 21 A and 21B show views of an embodiment of a brace garment for supporting a wrist joint of a wearer.

FIGS. 22A, 22B and 22C show different views of an embodiment of a brace garment for supporting an elbow joint of a wearer.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

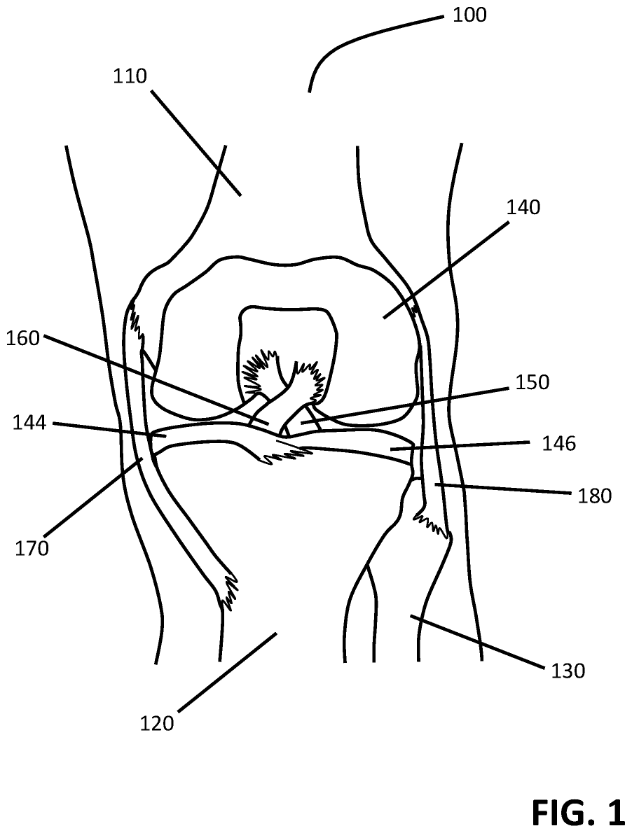

The present invention relates to a garment incorporating a brace arrangement for an articulating joint of a human body. The brace comprises tension members each disposed longitudinally along a corresponding predetermined curved three-dimensional spatial path within or on a matrix of the garment material, the corresponding predetermined paths spatially relating the one or more tension members to at least one natural ligament of the joint. In order to describe the garment and brace, we consider first the human knee as shown in FIG. 1.

FIG. 1 shows internal knee portion 100 of a human left leg as viewed from front or anterior, but with the patella or knee cap and its various adhesions removed for the sake of clarity of the internal structure of this particular joint. Major bones of the leg are shown as femur 110, tibia 120 and fibula 130. The portions of these bones that contact one another during articulation are provided with articular cartilage 140, the two portions of the cartilage on tibia 120 being medial meniscus 144 and lateral meniscus 146. The bones are stabilized with respect to one another by strategically placed ligaments that hold them together. These include posterior cruciate ligament (PCL) 150 and anterior cruciate ligament (ACL) 160 binding together the interacting faces of femur 110 and tibia 120, and medial collateral ligament (MCL) 170 and lateral collateral ligament (LCL) 180 binding respectively tibia 120 and fibula 130 to respectively the medial and lateral portions of the head of femur 110.

In the following description, various embodiments of an inventive garment are shown as disposed on a human from a middle section of the body and down the leg, with the entire lower portion of the body including the feet being covered by the garment. In actual embodiments, the garment may or may not extend all the way up to or past the waist, or completely covering the feet. Such variations in the configuration of a garment in accordance with the present invention may be dictated by style, manufacturing methods, and/or particular configurations for individual situations. Accordingly, the following detailed description of embodiments represents only a small fraction of the possible variations of such garments.

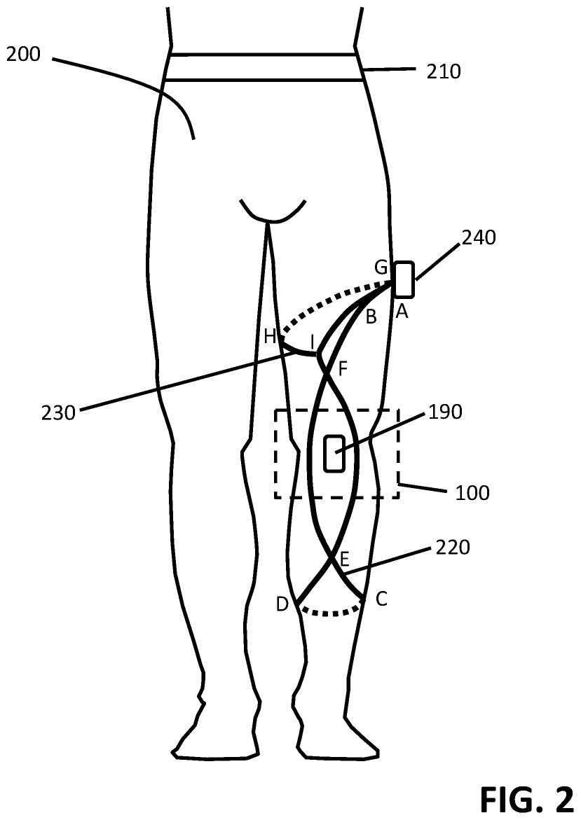

We turn now to a first embodiment of a brace garment shown in FIG. 2, in which the knee joint of FIG. 1 is dressed in garment 200 comprising belt 210, tension member 220, anchor member 230, and tensioner 240. Broken rectangle 100 indicates the region of the left leg shown in FIG. 1. Tension member 220 follows a predetermined path related to the disposition of ligaments 150, 160, 170 and 180 described above. Proceeding from tensioner 240 at point A, the path proceeds to point B where tension member 220 splits in its path to form a single closed loop path. The path proceeds from the left lateral side to pass patella 190 of the left leg on the medial side before curving around again to reach the lateral side of the left leg at point C at the distal portion of the triceps surae (the collection of muscles of the human calf), from where it proceeds around the back of the leg once more (shown in broken line) to the medial side of the left leg at point D. It then proceeds from point D to cross over its own path at point E in order to proceed along a curved path around the lateral side of patella 190. After crossing over itself at point F, the path curves sharply to the left lateral side to arrive at point B where it rejoins itself. At crossover points E and F, the outgoing and incoming portions of tension member 220 are independent, they do not join each other and are arranged to move as freely from each other as possible.

The path of anchor member 230 extends from tensioner 240 at point G around the back of the thigh (shown as a broken line) to point H on the medial side and then to point I where it terminates proximate tension member 220. The path of tension member 220 makes a sharp curve at point I. The exact way in which anchor member 230 and tension member 220 are arranged at point I is described in more detail below. For the present purposes it suffices to point out that the path of anchor member 230 and the path of tension member 220 jointly form a closed loop around the thigh to anchor brace garment 200 to the thigh. That is, with reference also to further embodiments below, tension member 220 and anchor member 230 together fully encircle the thigh irrespective of how any anchoring function is distributed between tension member 220 and anchor member 230. The loop from point C to point D to point E formed by the path of tension member 220 at the calf of the leg similarly anchors brace garment 200 to the calf.

In operation, tension member 220 may have its tension adjusted by the wearer of garment 200 via tensioner 240. Similarly, in embodiments in which anchor member 230 extends along its path within a conduit, as addressed in more detail below, anchor member 230 may have its tension adjusted by tensioner 240. In other embodiments, anchor member 230 may be comprised of a material that is longitudinally inextensible and tensioner 240 may in such embodiments be disposed on the inextensible material so that tensioning tension member 220 also tensions anchor member 230.

Tensioner 240 may be implemented in a variety of embodiments, including without limitation ratchet arrangements and lug screw arrangements. Any arrangement capable of establishing longitudinal tension in tension member 220 and compatible with human operation is suitable.

The embodiment of FIG. 2 specifically shows tension member 220 crossing over itself both above and below the knee. In a more general embodiment, it needs only cross over itself either above or below the knee. However, there is merit in the downward and upward portions of the path of tension member 220 approaching each other on the side of the knee opposing that on which the tension member 220 in fact does cross over itself.

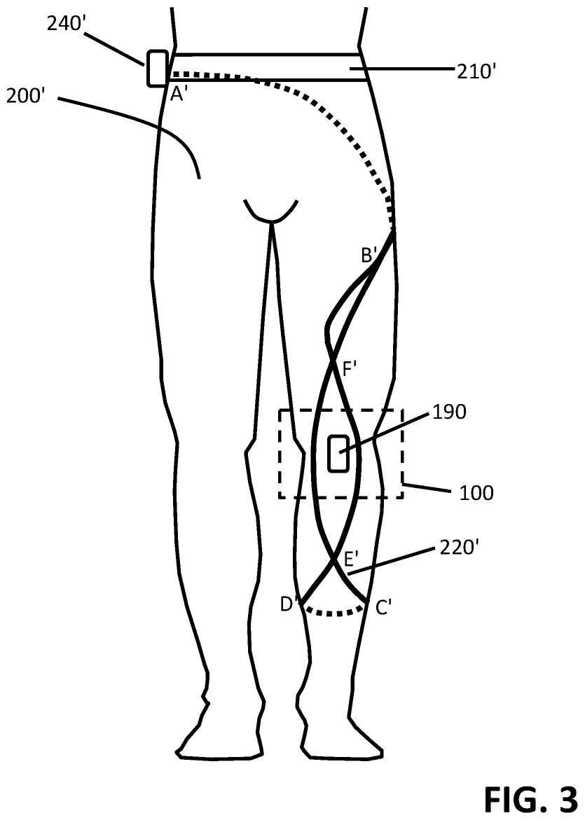

A second embodiment of a garment according to the present invention is shown in FIG. 3 as garment 200′ with belt 210′, tension member 220′, and tensioner 240′. In this embodiment, the schematic path of tension member 220′ starts at point A′ at tensioner 240′ on the right hip on belt 210′. It then proceeds from point A′ around the back of the wearer (shown in a broken line) to point B′, located on the lateral side of the left leg, where tension member 220′ splits in its path to form a single closed loop. The loop proceeds from the left lateral side to pass patella 190 of the left leg on the medial side before curving around again to reach the lateral side of the left leg at point C′ at the distal portion of the triceps surae, from where it proceeds around the back of the leg once more (shown in broken line) to the medial side of the left leg at point D′. It then proceeds from point D′, to cross over its own path at point E′, in order to proceed along a curved path around the lateral side of patella 190 before again crossing over its own path at point F′. The path then curves sharply to the left lateral side to arrive at the point B′, where it rejoins itself. At crossover points E′ and F′ the outgoing and incoming portions of tension member 220 are independent, they do not join each other and are arranged to move as freely from each other as possible.

In this arrangement, tension member 220′, by virtue of circling the leg, not only produces or exerts a stabilizing compressive force on knee region 100 but also produces or exerts forces on the leg and garment that allow the tension member to clamp the leg in region C′-D′-E′ around the calf. Tension member 220′ therefore serves as its own distal anchor to the leg and is anchored at the top in belt 210′ and by the fact that tension member 220′ completes at least a partial portion, perhaps a major portion, of a circle about the body. In operation, tension member 220′ may have its tension adjusted by the wearer of garment 200′ via tensioner 240′. In other embodiments, the tensioner may be located further around the anterior of the torso so that tension member 220′ completes a circle around the thigh as seen from the top down along the leg. In this disclosure, the phrase “fully encircles the thigh” is used to describe this latter situation, even though the actual path may be in the nature of a spiral. The segment of tension member 220′ extending from point A′ to point B′ in FIG. 3 may also be viewed as an anchor member, as it fulfills the same role as anchor member 230 in the embodiment of FIG. 2.

The embodiment of FIG. 3 specifically shows tension member 220′ crossing over itself both above and below the knee. In a more general embodiment, it needs only cross over itself either above or below the knee. However, there is merit in the downward and upward portions of the path of tension member 220′ approaching each other on the side of the knee opposing that on which tension member 220′ in fact does cross over itself.

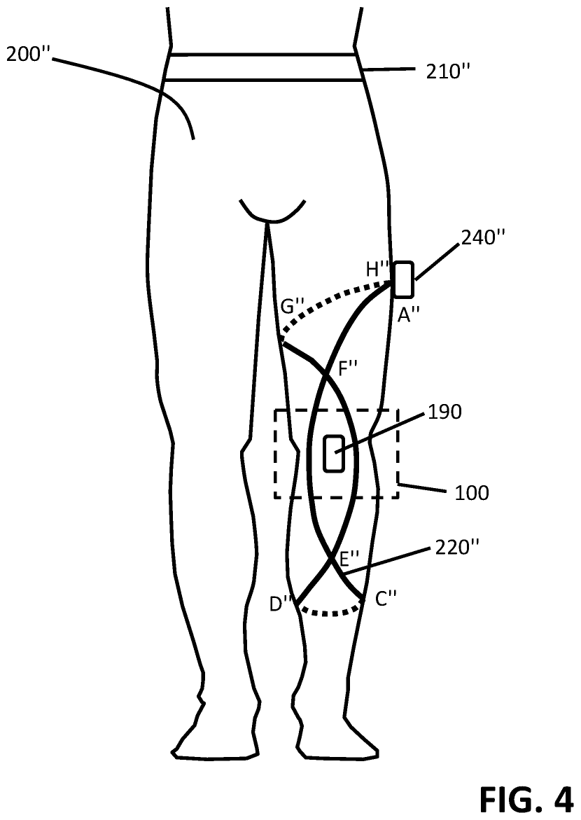

A further embodiment of the garment is shown in FIG. 4 as garment 200″ with belt 210″, tension member 220″, and tensioner 240″. In this embodiment, the schematic path of tension member 220″ starts at point A″ at tensioner 240″. It then proceeds from point A″ to pass patella 190 of the left leg on the medial side before curving around again to reach the lateral side of the left leg at point C″ at the distal portion of the triceps surae, from where it proceeds around the back of the leg (shown in broken line) to the medial side of the left leg at point D″. It then proceeds from point D″, to cross over its own path at point E″, in order to proceed along a curved path around the lateral side of patella 190 before again crossing over its own path at point F″. It curves around the medial side of the thigh at point G″ and proceeds around the back of the thigh over the posterior of the thigh (shown in broken lines) to tensioner 240″ at point H″. At crossover points E″ and F″ the outgoing and incoming portions of tension member 220″ do not join each other and are arranged to move as freely from each other as possible.

In this arrangement, tension member 220″, by virtue of circling the leg, not only produces or exerts a stabilizing compressive force on knee region 100 but also produces or exerts forces on the leg and garment that allow the tension member to clamp the leg in region C″-D″-E″ around the calf. Tension member 220″ therefore serves as its own distal anchor to the leg. Similarly, by virtue of circling the leg about the thigh it anchors the garment to the thigh. In operation, the tension member may have its tension adjusted by the wearer of garment 200″ via tensioner 240″.

The embodiment of FIG. 4 specifically shows tension member 220″ crossing over itself both above and below the knee. In a more general embodiment, it needs only cross over itself either above or below the knee. However, there is merit in the downward and upward portions of the path of tension member 220″ approaching each other on the side of the knee opposing that on which tension member 220″ in fact does cross over itself.

In the embodiments of FIG. 2, FIG. 3 and FIG. 4, tension member 220, 220′, 220″ in each case describes a tensionable loop, even if the tensionable loop is not circular and despite tensioner 240, 240′, 240″ being in the loop in some cases. Furthermore, in the various embodiments, tension member 220, 220′, 220″ and anchor member 230 separately or jointly either wholly, in major part, or at least partially encircle the thigh.

In the embodiments shown in FIG. 2 and FIG. 4, the garment need not assume the form of a complete lower body garment and need only cover the relevant portion of the leg being stabilized. The anchoring arrangements of FIG. 2 and FIG. 4 obviate belt 210, 210″. It is therefore possible for the embodiments shown in FIG. 2 and FIG. 4 to be implemented in the form of a hose for the affected leg, the hose extending from the upper thigh to below the calf. Wearers may prefer to employ a more extensive garment for aesthetic reasons. Even if it assumes the general aspect of a complete lower body garment, the self-anchoring aspect of tension member 220, 220″ makes it possible for the garment not to have to extend to or over the feet. Garment 200, 200′, or 200″ may envelope the knee wholly, or only in part. For example, the posterior of the knee or the patella may in principle remain uncovered by garment 200, 200′ or 200″.

The choice of the exact path of tension member 220, 220′, 220″ is dictated by the forces required for stabilizing the knee, which in turn is dictated by the support and compression desired for the four ligaments in the knee discussed above at the hand of FIG. 1. In this respect it will be noted that tension member 220, 220′, 220″ passes proximate the two lateral ligaments MCL and LCL to compress the knee in those areas. The path of tension member 220, 220′, 220″ is therefore predetermined by the location of the ligaments. In FIG. 2, FIG. 3 and FIG. 4, a single tension member is employed disposed longitudinally along a predetermined path. That is, the tension is directed longitudinally along the path. The garment may in general comprise one or more tension members each disposed longitudinally along a corresponding predetermined curved three-dimensional spatial path within or on a matrix of a garment material, the corresponding predetermined paths spatially relating the one or more tension members to at least one natural ligament of the anatomical joint.

We turn now to the nature of tension member 220, 220′, 220″ and its interaction with the matrix of the material from which garment 200, 200′, 200″ is fashioned. FIG. 2, FIG. 3 and FIG. 4 schematically show the paths of tension members 220, 220′, 220″, but not how tension member 220, 220′, 220″ is housed or retained in or on garment 200, 200′, 200″. Tension members 220, 220′, 220″ have to be free to move within garment 200, 200′, 200″ without crumpling or significantly distorting the garment. To achieve this, tension members 220, 220′, 220″ are routed along their paths within conduits in the matrix of the material from which garment 200, 200′, 200″ is fashioned. The conduits may comprise natural passages within the weave of the material of the garment. In other embodiments, the conduits may be tubular in nature and may be disposed within or external to but attached to the material matrix of garment 200, 200′, 200″. In yet further embodiments, the conduits may comprise segments of tubing, also described in the present disclosure by the term “collated tubing”. The use of collated tubing allows the degree of friction between tension member 220, 220′, 220″ and the matrix material of garment 200, 200′, 200″ to be modified. In yet further embodiments, the conduits may be fashioned from a clothing material different from that of the matrix of garment 200, 200′, 200″. In yet further embodiments, the conduits may be fashioned by a stitching pattern that demarcates the tension member path either side of tension member 220, 220′, 220″. A special case of such a stitched arrangement is one in which the material of the garments is stitched together in a seam, and tension member 220, 220′, 220″ extends longitudinally within the seam.

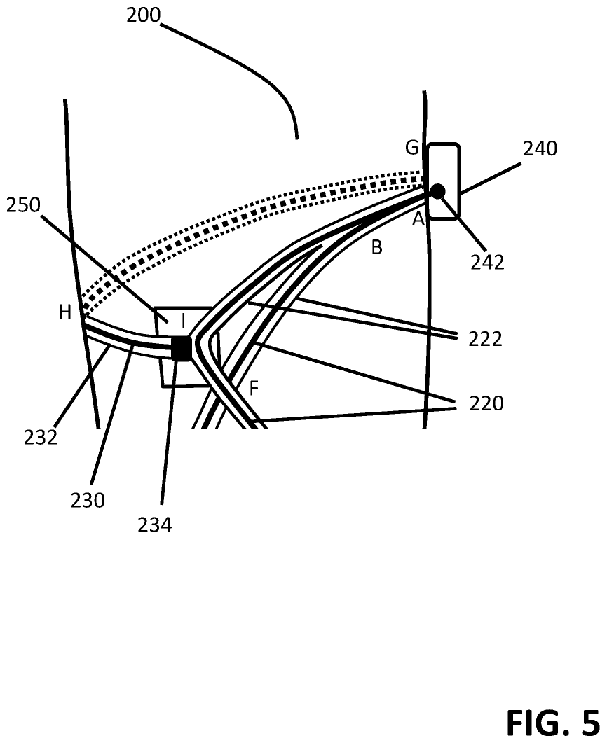

Tension member 220, 220′, 220″ may comprise a plurality of individual strands extending along individual conduits, the individual strands being joined together before entering tensioner 240, 240′, 240″. Tension member 220, 220′, 220″ may be formed of a material that is substantially less extensible than the material matrix of the garment. Materials suited for use in tension member 220, 220′, 220″ may be longitudinally inextensible but flexible. Suitable materials for tension member 220, 220′, 220″ include, but are not limited to, stainless steel; Nylon; Kevlar®; Teflon®; ultra-high molecular weight polyethylene-based fiber such as Dyneema®; and various fibers with a diamond or basket weave including cotton, polyester and polypropylene, for example without limitation Technora®. (Kevlar is a registered trademark of E. I. DU PONT DE NEMOURS AND COMPANY CORPORATION DELAWARE Chestnut Run Plaza, 974 Centre Road WILMINGTON DELAWARE 19805; Teflon is a registered trademark of THE CHEMOURS COMPANY FC, LLC LIMITED LIABILITY COMPANY DELAWARE 1209 ORANGE STREET WILMINGTON DELAWARE 19801; Dyneema is a registered trademark of DSM IP Assets B.V. LIMITED LIABILITY COMPANY NETHERLANDS Het Overloon 1 HEERLEN NETHERLANDS NL6411 TE; and Technora is a registered trademark of TEUIN KABUSHIKI KAISHA (TEUIN LIMITED) CORPORATION JAPAN 6-7, MINAMIHOMMACHI 1-CHOME CHUO-KU, OSAKA JAPAN) The conduit may be formed of the same material as the matrix material of the garment and be lined internally with a conduit material of lesser friction coefficient with respect to the tension member material so as to allow the tension member 220, 220′, 220″ to move as freely as possible. The conduit material may be in the form of a tube or a collated tube. There is no particular structural limitation on the cross-section of the tube. A collated tube is useful in that it allows a very low friction tube material to be selected even if it is inextensible, because the collation segments may move closer to one another without unduly crumpling the garment 200, 200′, 200″ when tension member 220, 220′, 220″ is put under tension. As shown in FIG. 2, FIG. 3, FIG. 4 and FIG. 5, tensioner 240, 240′, 240″ may be viewed as variously in or on the loop formed by the tension member 220, 220′, 220″.

Considering tension members 220, 220′, 220″ as freely moving within their respective conduits, we now return to FIG. 2 in order to elucidate the matter of the relationship between anchor member 230 and tension member 220 at point I. To this end, the thigh region of FIG. 2 is shown enlarged and with more detail in FIG. 5. In one embodiment, anchor member 230 may extend from point G to point I along conduit 232 similar to conduit 222 of tension member 220. In order for this latter arrangement to function as an anchor, anchor member 230 is attached to end 234 of conduit 232 proximate point I. It is to be noted that anchor member 230 is not attached to tension member 220 at point I, because tension member 220 needs to remain free to move within conduit 222. In this arrangement, the portion of tension member 220 extending from point A at tensioner 240 to point I, together with the whole of anchor member 230 form an anchor that extends around the thigh. As tension member 220 needs to be able to move freely past point I, the curvature of the path of tension member 220 at point I has an upper curvature limit dictated by the flexibility of tension member 220. This favors a materials choice for tension member 220 that requires the material to be substantially longitudinally inextensible, yet highly flexible. This makes a variety of fibers with a diamond or basket weave including cotton, polyester and polypropylene useful for the particular embodiment in FIG. 2 and FIG. 5.

Any portion 250 of garment 200 between anchor member 230 and tension member 220 may be made from inextensible material to ensure that any tension applied to anchor member 230 by tensioner 240 does not simply stretch portion 250 of garment 200 proximate the region between anchor member 230 and tension member 220. In other embodiments, the end of anchor member 230 near point I may be shaped into a loop in order to spread the force acting at the end of anchor member 230 over a larger area to avoid pulling or deforming the fabric of garment 200. FIG. 5 also shows tension member 220 entering tensioner 240 via tensioner port 242.

There is much freedom in the choice of the matrix material or fabric of the garment 200, 200′, 200″. There is some merit in using a material with considerable elasticity, for example without limitation Spandex or mixes of Spandex with other fibers, including for example without limitation cotton. This allows the garment to be made with an intentionally tight fit. This reduces the possibility of crumpling when tension member 220, 220′, 220″ is put under tension.

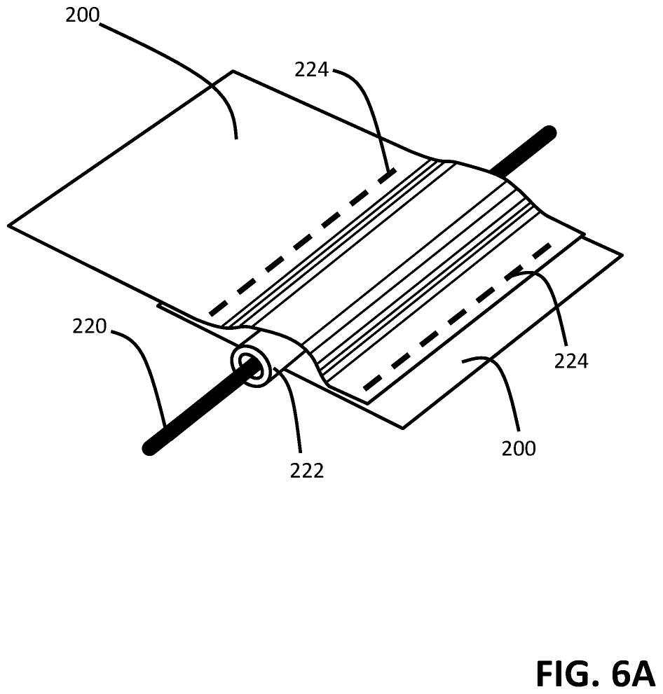

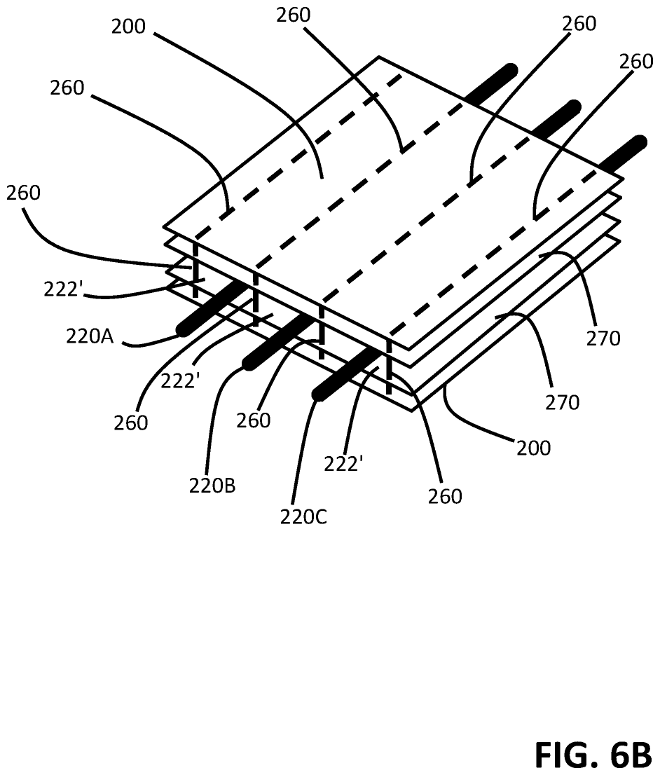

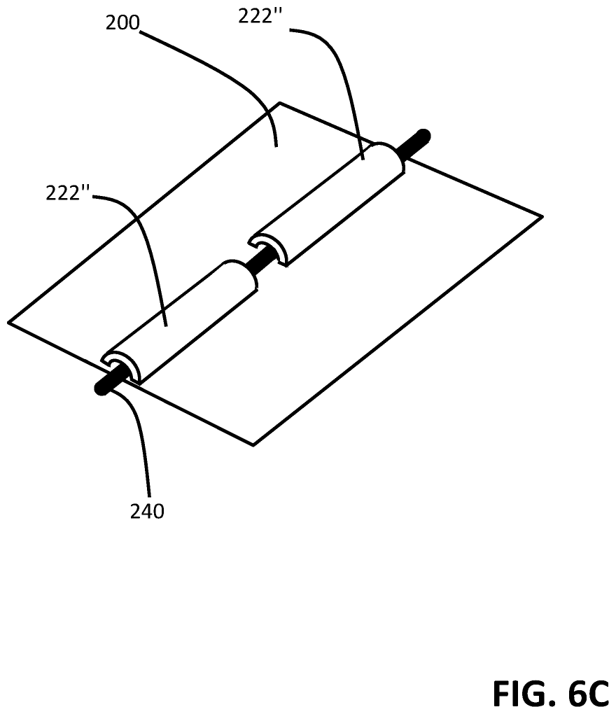

FIG. 6A, FIG. 6B, and FIG. 6C show different implementations of conduits for use with tension members. Using the elements of FIG. 2 and FIG. 5 as example, FIG. 6A shows a seam in garment 200 material matrix created by stitches 224, with conduit 222 extending along the seam in the form of tube in this embodiment. Tension member 220 extends longitudinally through conduit 222. The material of conduit 222 is selected to have a low coefficient of friction with tension member 220. In a more general case, there may be a plurality of seams, each having conduit 222 extending longitudinally along it and each conduit 222 having a strand of tension member 220 extending longitudinally along it. In FIG. 6A, conduit 222 is shown as circular in cross-section, but in other embodiments it may have any suitable cross-section that allows the strand of tension member 220 to move substantially freely, while simultaneously making conduit 222 compatible with the ergonomic requirements to which the garment is subject. One suitable cross-section is semi-circular, or a smaller segment of a circle with enough curvature to accommodate the strand of tension member 220, while being flat on one side so as to be easily integrated in garments material 200. It has already been explained that conduit 222 may be collated, which allows very low friction, but inextensible tubing to be employed. Example materials for the tubing include, but are not limited to Teflon® and silica. In a related embodiment, the strand of tension member 220 is simply sandwiched between two strips of low fiction material inside a stitched seam. In a further embodiment, seams may be created by joining adjacent material matrices by an adhesive, heat sealing, or other conjoining mechanism, and a conduit formed between suitable spaced-apart seams. Conduits may be similarly implemented in the embodiments of FIG. 3 and FIG. 4.

FIG. 6B shows another embodiment of a way to incorporate tension member 220 within the matrix material of garment 200. The drawing is an edge-on view of the weave of garment 200. It shows four rows 260 of stitching or weave either side of every one of three strands of tension member 220 within the matrix material of garment 200. The three strands of tension member 220, being 220A, 220B, and 220C, are sandwiched between two longitudinal strips 270 of low friction material held by stitching 260. In this drawing, the material of garment 200 is shown as being two sheets. This should be considered as presented schematically for the sake of clarity, as the material is in practice woven. The fiber of the stitching may be a low friction material so that tension member 220 slides on all sides against low friction material. In this embodiment, conduits 222′ are formed by low friction material strips 270 and low friction material stitching 260. This embodiment may be implemented at seams in garment 200 or the sandwiching of garment material 200 with low friction material 270 may be implemented over the entire area of tension member 220. The conduits may be similarly implemented in the embodiments of FIG. 3 and FIG. 4.

FIG. 6C shows an embodiment in which conduit 222″ is attached to the surface of garment 200 in the form of collated semi-cylindrical tubes and tension member 220 routed through conduit 222″. In some embodiments, the bases of the tubes may be closed or may be lined with a low friction material. In FIG. 6A, FIG. 6B, and FIG. 6C tension member 220, 220′, 220″ is shown as circular in profile or cross-section. In a general embodiment, there is no limitation on the profile or cross-section of the tension member 220, 220′, 220″.

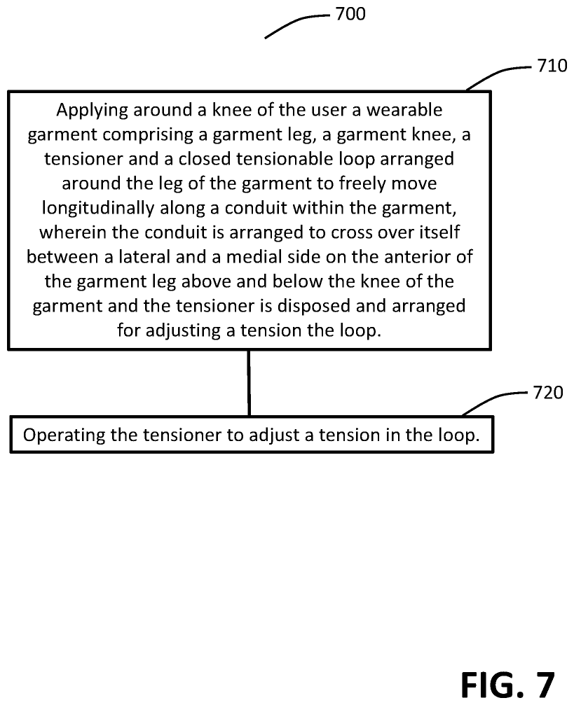

In a further aspect, shown in the flow chart of FIG. 7, method [700] is presented for stabilizing a knee of a human user leg, comprising: applying [710] around a knee of the user wearable garment 200, 200′, 200″ comprising a garment leg, a garment knee, tensioner 240, 240′, 240″ and closed tensionable loop 220, 220′, 220″ arranged around the leg of garment 220, 220′, 220″ to freely move longitudinally along conduit 222, 222′, 222″ within or on garment 200, 200′, 200″, wherein conduit 222, 222′, 222″ is arranged to cross over itself between a lateral and a medial side on the anterior of the garment leg at least one of above (point F, F′, F″) and below (point E, E′, E″) the knee of garment 200, 200′, 200″ and tensioner 240, 240′, 240″ is disposed and arranged for adjusting tension of loop 220, 220′, 220″; and operating [720] tensioner 240, 240′, 240″ to adjust tension in loop 220, 220′, 220″. In some embodiments garment 200, 200′, 200″ may be applied such that conduit 222, 222′, 222″ is arranged to cross over itself between a lateral and a medial side on the anterior of the garment leg both above (point F, F′, F″) and below (point E, E′, E″) the knee of garment 200, 200′, 200″.

Applying [710] may comprise: arranging garment 200, 200′, 200″ on the user to position a first portion of loop (C, C′, C″ to D, D′, D″) against a distal region of a triceps surae of the user leg; positioning a second portion of the loop (the portion at above point F, F′, F″) on or above a thigh of the user leg; and positioning on the medial and lateral sides of a patella of the user leg proximate two collateral ligaments of the user knee third and fourth portions of the loop located on the loop between the first and second portions (both between E, E′, E″ and F, F′, F″). The positioning of the second portion of the loop may comprise positioning the second portion of the loop against the posterior of the thigh of the user leg (see FIG. 4).

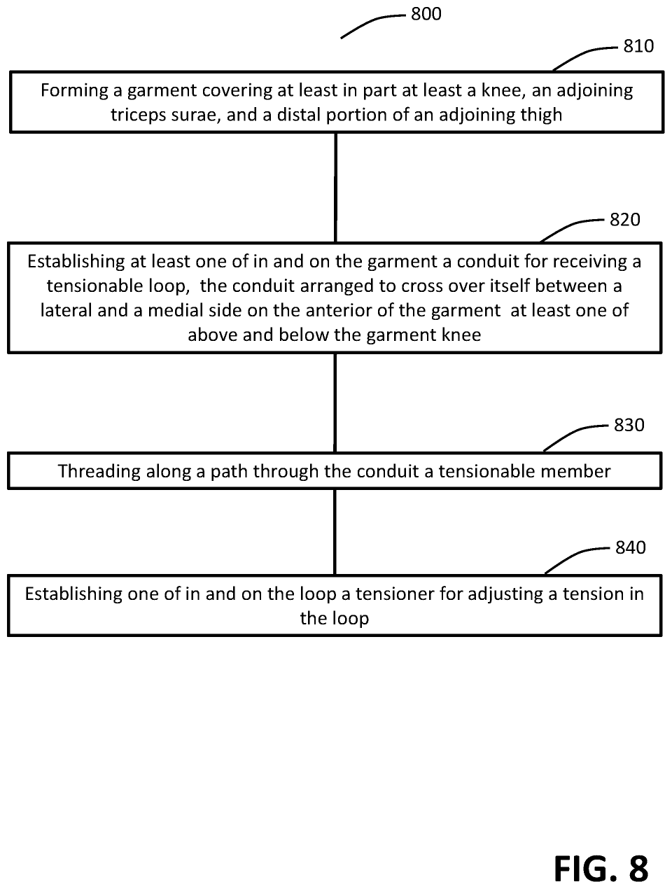

In a further aspect, described at the hand of the flow chart of FIG. 8, method [800] is presented for manufacturing a wearable garment for stabilizing a knee of a user, the method comprising: forming [810] a garment covering at least in part at least a knee, an adjoining triceps surae, and a distal portion of an adjoining thigh; establishing [820] at least one of in and on the garment a conduit for receiving a tensionable loop, the conduit arranged to cross over itself between a lateral and a medial side on the anterior of the garment at least one of above and below the garment knee; threading [830] along a path through the conduit a tensionable member; establishing [840] one of in and on the loop a tensioner for adjusting a tension the loop.

Threading [830] the tensionable member may comprise threading a tensionable member that is substantially inextensible. Threading [830] the tensionable member may comprise threading a tensionable member that is flexible and substantially inextensible. Threading [830] the tensionable member may comprise threading a tensionable member made from one or more of PTFE, stainless steel, Nylon; Kevlar®; one or more ultra-high molecular weight polyethylene-based fiber, a fiber with a diamond weave. Threading a tensionable member made from the fiber with a diamond weave may comprise threading a tensionable member made from one of cotton, polyester, polypropylene, and Technora®.

Establishing the conduit [840] may comprise arranging the conduit to cross over itself above and below the knee. Forming the garment [810] may comprise extending the garment to a waist of the user, forming a belt around the waist. Establishing the tensioner may comprise establishing the tensioner on the belt. Establishing [840] the tensioner in or on the loop may comprise establishing the tensioner on a thigh of the garment.

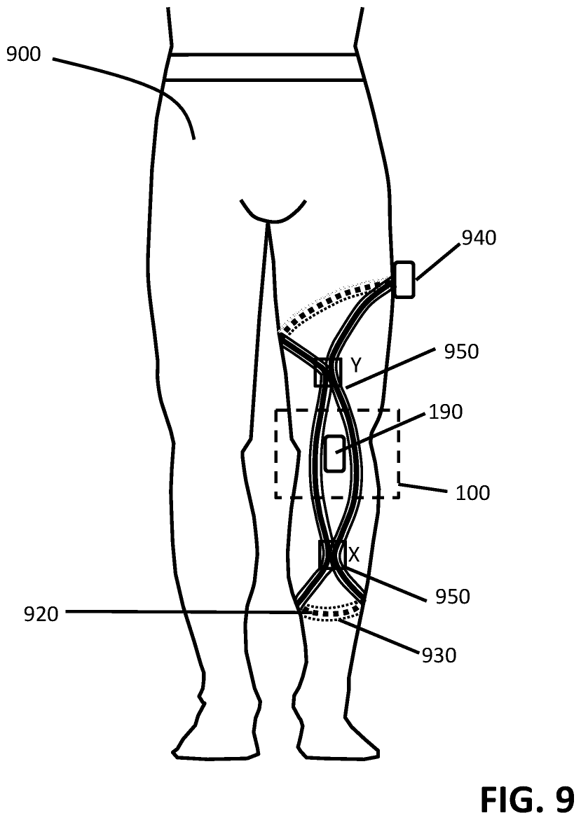

In a further embodiment, shown in FIG. 9, a wearable garment 900 is presented for stabilizing knee 100 of a user comprising: closed tensionable loop 920 arranged to freely move along conduit 930 disposed within or on garment 900, conduit 930 extending along a conduit path having relative to knee 100 a lateral portion and a medial portion; and adjustable tensioner 940 in or on loop 920 for adjusting a tension in loop 920, wherein the lateral and medial portions of the conduit path pass patella 190 of user knee 100 proximate lateral 170 and medial 180 collateral ligaments of user knee 100 in FIG. 1 respectively and in which the lateral and medial portions of the conduit path closely approach each other at one or more than one point, the at least one point being at least one of below (point X) and above (point Y) user knee 100. Broken lines in FIG. 9 indicate that the conduit and tensionable loop are on the posterior of the leg.

The lateral and medial portions of the conduit path may closely approach each other at both a first point (point X) below user knee 100 and at a second point (point Y) above user knee 100. It should be noted that, while the actual conduits portions may overlap at point X and point Y, the lateral and medial portions of the tensionable loop inside the conduit are not joined at point Y or point X. Tensionable loop 920 may be disposed for applying pressure to at least one of lateral collateral ligament 170 and medial collateral ligament 180 of the user when garment 900 is worn by the user and the tensionable loop is tensioned by operating tensioner 940. Conduit 930 may be arranged along a path that substantially circles the leg at a distal region of the triceps surae of the leg, as shown in FIG. 9. This anchors garment 900 to the triceps surae. Tensionable loop 920 may comprise a tension member extending along conduit 930. Tensioner 940 may be disposed on the thigh of the user. The tension member may be made of a substantially longitudinally inextensible material and the material may be flexible. Loop 920 may be arranged in or on the material of garment 900 in the same way as shown in FIG. 6A, FIG. 6B and FIG. 6C and in the text accompanying those drawings. Conduit 930 may comprise a low friction tube and the tube may be collated. As with the embodiment of FIG. 4, tensionable loop 920 in FIG. 9 extends substantially around the thigh, thereby anchoring garment 900 to the thigh.

The anchoring arrangements on the thigh of the user employed in the embodiments shown in FIG. 2, FIG. 3, and FIG. 4 may also be applied to the embodiment in FIG. 9. In order to ensure that the material of garment 900 does not deform and stretch and thereby disrupt the forces applied by tensionable loop 920, portions 950 of the garment may be made from suitable inextensible materials. The embodiment of FIG. 9 may be viewed as a very similar to the case of FIG. 4, but without loop 920 overlapping itself.

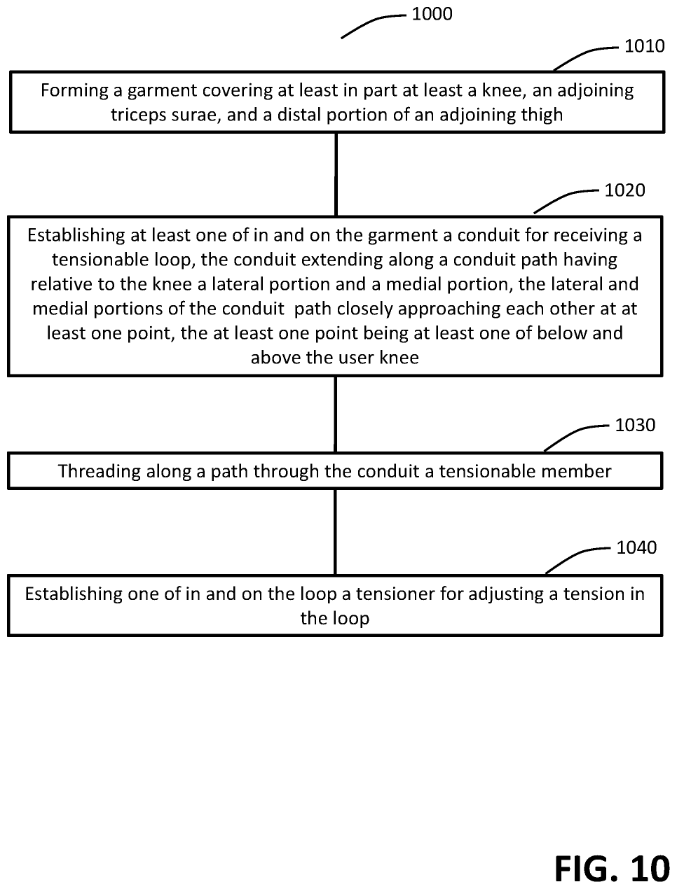

In a further aspect, described at the hand of FIG. 10, method [1000] is presented for manufacturing wearable garment 900 for stabilizing knee 100 of a user, the method comprising: forming [1010] garment 900 covering at least in part at least knee 100, an adjoining triceps surae, and a distal portion of an adjoining thigh; establishing [1020] at least one of in and on garment conduit 930 for receiving tensionable loop 920, conduit 930 extending along a conduit path having relative to knee 100 a lateral portion and a medial portion, the lateral and medial portions of the conduit path closely approaching each other at least one point, the at least one point being at least one of below and above user knee 100; threading [1030] along a path through conduit 930 a tensionable member; establishing [1040] one of in and on loop tensioner 940 for adjusting a tension in loop 920.

Threading [1030] the tensionable member may comprise threading a tensionable member that is substantially inextensible. Threading [1030] the tensionable member may comprise threading a tensionable member that is flexible and substantially inextensible. Threading [1030] the tensionable member may comprise threading a tensionable member made from one or more of PTFE, stainless steel, Nylon Kevlar®; one or more ultra-high molecular weight polyethylene-based fiber, a fiber with a diamond weave. Threading a tensionable member made from the fiber with a diamond weave may comprise threading a tensionable member made from one of cotton, polyester, polypropylene, and Technora®. Establishing [1020] conduit 930 may comprise arranging conduit 930 to have the lateral and medial portions of the conduit path closely approaching each other both below and above user knee 100. Forming [1010] garment 900 may comprise extending garment 900 to a waist of the user, forming a belt around the waist, in which establishing tensioner 940 comprises establishing tensioner 940 on the belt. Establishing [1040] tensioner 940 in or on the loop may comprise establishing the tensioner on a thigh of the garment.

In a variant of the embodiment of FIG. 4, a portion of tension member 220″ may comprise a length of substantially longitudinally inextensible fabric incorporated within the fabric of garment 200″. In FIG. 4, the relevant portion of tension member 220″ composed of substantially longitudinally inextensible material may be, for example, segment G″ to H″. The remaining portion of tension member 220″ may comprise of inextensible strands within conduits as already described above. The inextensible strands are anchored to the segment G″-H″ at both of its ends. Tensioner 220″ may be mounted on or at one end of the segment G″-H″. In this variant of the embodiment in FIG. 4, tensioner 220″ is mounted at end H″ of segment G″-H″ of substantially longitudinally inextensible material. In the same manner, portions of tension members 220, 220′, 920 of the embodiments of FIG. 2, FIG. 3 and FIG. 9 may be comprised of substantially longitudinally inextensible material.

In the present specification, the term “substantially inextensible fabric” is used to describe a fabric extending in two dimensions that is substantially inextensible in at least a first direction. The fabric may or may not have a restriction on extensibility in a direction perpendicular to the first direction. Non-limiting examples of such materials include but are not limited to so-called 2-way-stretch fabrics with blends of materials including Spandex, Nylon, Dyneema®, Kevlar®, polyester, Ingeo®, olefin fibre, Lyocell, and/or cotton which are woven, knitted, or braided in such a fashion to allow stretch in required dimensions. (Ingeo is a registered trademark of NATUREWORKS LLC LIMITED LIABILITY COMPANY DELAWARE 15305 MINNETONKA BLVD MINNETONKA MINNESOTA 55345). The “2-way” stretching refers here to stretching in two opposing directions in a first dimension whilst remaining substantially inextensible in any direction perpendicular to the first direction. Other “substantially inextensible fabrics”, including for example without limitation materials comprising Dyneema® fibers in a two-dimensional mesh embedded in a second fabric or in a sandwich structure, may have substantially no extensibility in any direction. The inextensibility is deemed “substantial” in comparison with the inextensibility of the matrix material of the fabric of garment 200″, the garment fabric being stretchable or extensible in comparison with the “substantially inextensible fabric”.

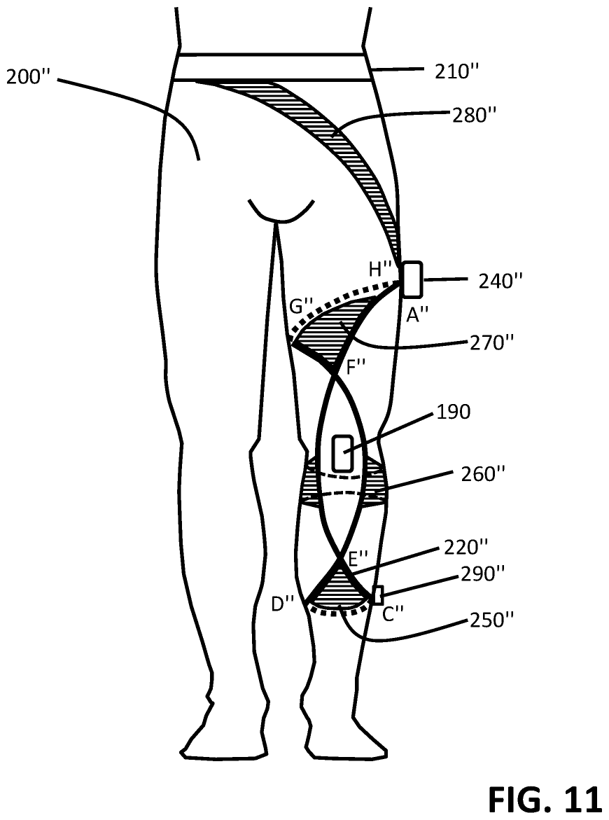

A further aspect of the wearable garment for stabilizing knee 100 of the user may be best described at the hand of FIG. 11, in which a variant of the garment of FIG. 4 is shown. The numbering of FIG. 4 is maintained in FIG. 11. Stabilizing connector 280″ connecting tension member 220″ to belt 210″ serves to keep garment 200″ from being pulled down as the tension in tension member 220″ is adjusted. Stabilizing connector 280″ may be implemented in many different ways, all of the implementations sharing the principle of connecting tension member 220″ to belt 210″. In FIG. 11 stabilizing connector 280″ is implemented as a belt of substantially inextensible fabric exhibiting inextensibility in at least the longitudinal direction in order to maintain tension in that direction. In FIG. 11, stabilizing connector 280″ is shown as extending across the front of garment 200″, but it may also extend simultaneously from tension member 220″ to belt 210″ across the rear of garment 200″. In other embodiments, stabilizing connector 280″ may comprise a substantially inextensible tensionable member disposed within a conduit and may therefore be of the same construction as tension member 220″.

In order to help ensure that garment 200″ is not unduly deformed or crumpled when the tension in tension member 220″ is adjusted, stabilizing portions 250″, 260″, and or 270″ of substantially inextensible fabric are incorporated in the fabric or matrix material of garment 200″ and attached to conduit 222 (see FIGS. 6A, 6B and 6C) of tension member 220″ or to the garment material or fabric immediately adjacent to conduit 222. The substantially inextensible fabric used for stabilizing portions 250″, 260″, and or 270″ is employed with at least the horizontal selected to be the inextensible direction.

Stabilizing portion 250″ on the anterior of the leg below and proximate crossover point E″ exerts on segments E″-C″ and E″-D″ of tension member 220″ forces with major components perpendicular to the paths of tension member 220″ along those two segments E″-C″ and E″-D″. Stabilizing portion 250″ may be fastened to the conduit bearing tension member 220″ along those two segments E″-C″ and E″-D″. In FIG. 11, stabilizing portion 250″ is shown as extending in a generally triangular shape over most of the anterior of the leg below crossover point E″ between segments E″-C″ and E″-D″. In general, stabilizing portion 250″ may extend over any fraction of the anterior of the leg below crossover point E″ between segments E″-C″ and E″-D″ in order to establish a lateral tension below crossover point E″ between segments E″-C″ and E″-D″ over the anterior of the leg.

Stabilizing portion 270″ may be disposed on the anterior of the leg above and proximate crossover point F″ and exerts on segments F″-A″ and F″-G″ of tension member 220″ forces with major components perpendicular to the paths of tension member 220″ along those two segments F″-A″ and F″-G″. Stabilizing portion 270″ may be fastened to the conduit bearing tension member 220″ along those two segments F″-A″ and F″-G″. In FIG. 11, stabilizing portion 270″ is shown as extending in a generally triangular shape over most of the anterior of the leg above crossover point F″ between segments F″-A″ and F″-G″. In general, stabilizing portion 270″ may extend over any fraction of the anterior of the leg above crossover point F″ between segments F″-A″ and F″-G″ in order to establish a lateral tension above crossover point F″ between segments F″-A″ and F″-G″ over the anterior of the leg.

Stabilizing portion 260″ over largely the posterior of the leg above and proximate crossover point F″ exerts on the medial and lateral segments E″-F″ of tension member 220″ forces with major components perpendicular to the paths of tension member 220″ along those lateral and medial segments E″-F″. Stabilizing portion 260″ may be fastened to the conduit bearing tension member 220″ along those lateral and medial segments E″-F″. In FIG. 11, stabilizing portion 260″ is shown as specifically extending in a generally rectangular shape over a portion of the posterior of the leg above crossover point E″ and below patella 190 between the lateral and medial segments E″-F″. In general, stabilizing portion 260″ may extend over any portion of the posterior of the leg below crossover point F″ and above crossover point E″ between lateral and medial segments E″-F″ in order to establish a lateral tension below crossover point F″ and above crossover point E″ between lateral and medial segments E″-F″ over the posterior of the leg. Consideration of the need for freedom of articulation of the knee may restrict the region between crossover points E″ and F″ that is covered by stabilizing portion 260″, leading to the example arrangement shown in FIG. 11.

In a general embodiment, stabilizing portions 250″, 260″, and or 270″ are comprised of fabric that is substantially inextensible in a generally horizontal direction and stabilizing portions 250″, 260″, and or 270″ are disposed to extend between horizontally opposing medial and lateral portions of tension member 220″, the lateral and medial portions of tension member 220″ exhibiting lateral tension with respect to each other in the garment when tension member 220″ is placed under longitudinal tension by operating tensioner 240″. As may be understood from FIG. 11, the lateral tension is induced around the posterior of the leg between crossover points E″ and F″ in the general area of patella 190, while below and above crossover points E″ and F″ the lateral tension is induced over the anterior of the leg. In the general case, garment 200″ may comprise a portion of inextensible fabric laterally joining two segments of tension member 220″ in tension with respect to each other horizontally over the leg.

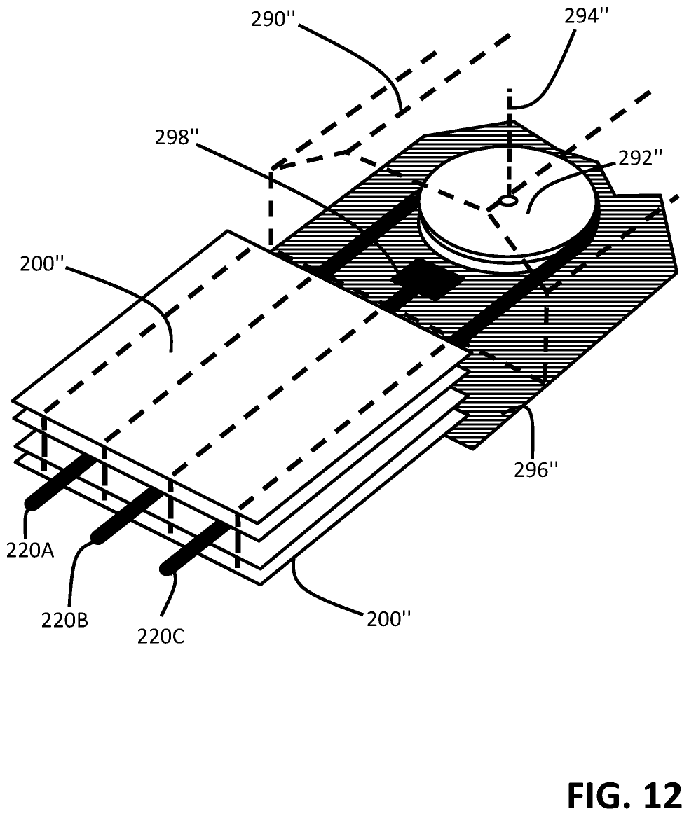

In a further aspect, described at the hand of FIG. 12 with reference to FIG. 11 and FIG. 6B, garment 200″ may comprise tension regulator 290″ to balance the tension between at least two strands within tension member 220″. By way of example we may consider tension member 220″ as comprising of a plurality of strands 220A, 220B, and 220C as in FIG. 6B. In FIG. 12 we show one end of tension member 220″, and thereby one end of strands 220A, 220B, and 220C. By way of non-limiting example, the end of tension member 220″ shown in FIG. 12 may be proximate tension regulator 290″ (see FIG. 11) disposed at the distal end of tension member 220″. When the garment is worn by the user and the legs move, tension member 220″ flexes and, in the absence of regulator 290″, may lead to significant differences in tension in different strands of tension member 220″. The presence of regulator 290″ allows tension member 220″ to flex without inducing unbalanced tensions that might disturb the functioning of garment 200″ and the comfort of the user by warping tension member 220″.

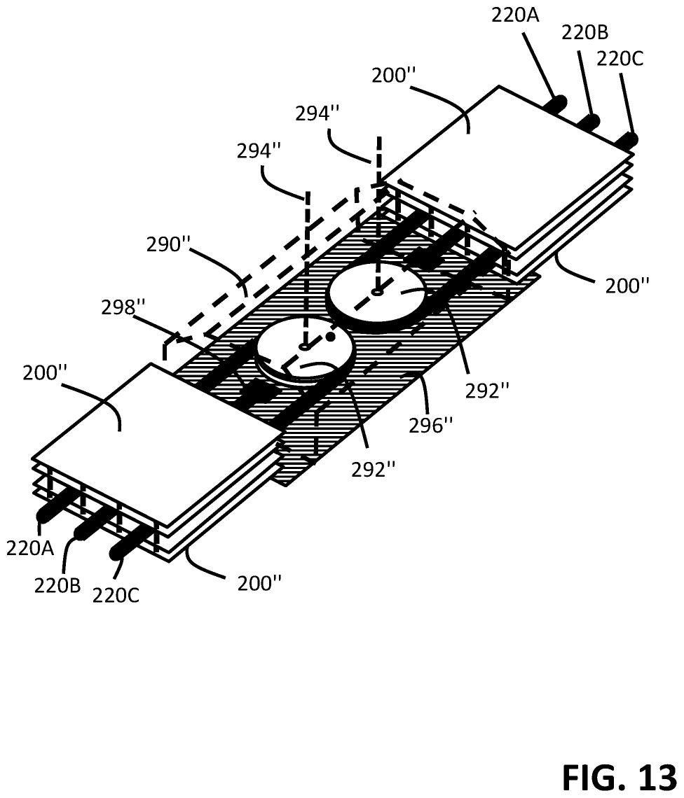

In FIG. 11, tension regulator 290″ is shown as disposed on the lateral side of the left leg at point C″ and comprises two ends of tension member 220″. In alternative embodiments, regulator 290″ may be placed elsewhere in tension member 220″, such as, for example, above point F″. Within regulator 290″, as shown in more detail in FIG. 12, two outer strands 220a and 220c may be joined in order to form a single strand arranged to move freely on pulley 292″. For the sake of clarity, some of the numbering used in FIG. 6 has been removed in FIG. 12 and only one end of tension member 220″ is shown, the other end being identical but rotated by 180 degrees with respect to the one shown. Pulley 292″ may rotate about pulley axis 294″. Tension regulator 290″ may be disposed on base 296″ of substantially inextensible fabric with an inextensible direction parallel to strands 220A, 220B, and 220C of tension member 220″. When tension member 220″ comprises an odd number of strands, as in FIG. 12, the unpaired central strand may be terminated proximate or in tension regulator 290″. Various ways are contemplated for terminating central strand 220B, and in FIG. 12 this is shown as general termination 298″. FIG. 13 shows how both ends of tension member 220″ may be terminated within tension regulator 290″, with two pulleys 292″ arranged head-to-head to balance the tension between strands 220A and 220C at the two ends of tension member 220″. While a tension regulator at one end of tension member 220″ may have the only one pulley 292″, the fact that tension regulator 292″ of FIG. 11 has to regulate tension at two ends of tension member 220″, implies that tension regulator 292″ of FIG. 11 has the two-pulley arrangement of tension regulator 292″ of FIG. 13.

FIGS. 6B and 12 show only one pair of joined strands within tension member 220″, but in general there may be a plurality of pairs of strands joined in this fashion. Such strands may be arranged in the generally symmetrical fashion shown in FIG. 12, each pair with a suitable pulley to balance the tension between the two joined strands in each pair. FIG. 12 shows on odd number of strands, namely three. In other embodiments the total number of strands in tension member 220″ may be even, so that there are only joined pairs of strands. In general, the strands in the pairs are arranged to be joined such that the pairs are generally substantially mirror symmetrically disposed about the center line of the overall tension member. Each pair balances its tension between its two constituent strands by moving around a suitable pulley as described above.

In other embodiments, the regulator may have any other mechanical arrangement that allows the tension in two strands in a pair roughly equidistant from the center line of tension member 220″ to be balanced. This includes, by way of non-limiting example, a curved tube (not shown) that allows the joined strands to slide freely within the curved tube.

In other embodiments, tension regulator 290″ may be disposed proximate tensioner 240″. In yet further embodiments, tension regulator 290″ may be integrated into tensioner 240″. In such an embodiment, the two pulley arrangements of FIG. 13 may be employed with the following modification. The regulating function is maintained by the rotation of the strands about the pulleys, while the tension may, by way of non-limiting example, be adjusted by moving two pulley axes 294″ toward or away from each other to respectively increase and decrease the tension. This allows the arrangement of FIG. 13 to function as an integrated combination of a tensioner and a tension regulator.

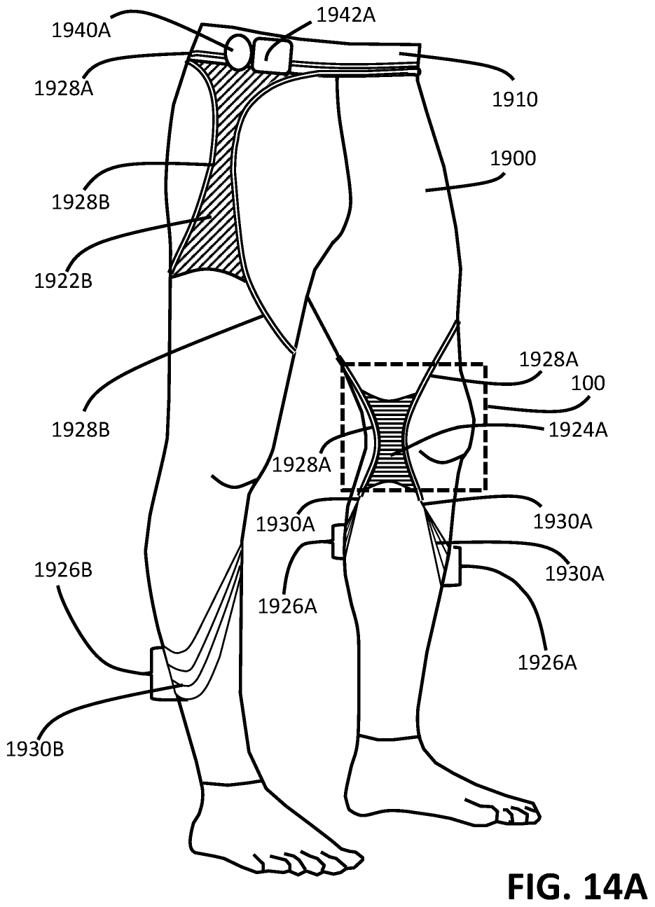

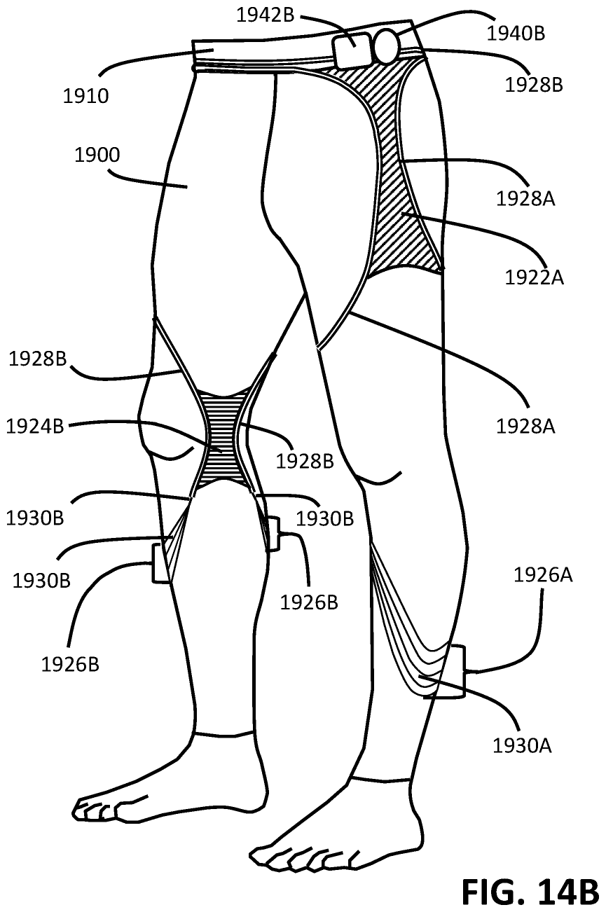

In a further embodiment, shown in FIG. 14A and FIG. 14B, the left knee joint of FIG. 1 is addressed by medial collateral ligament brace garment 1900 comprising belt 1910, tensioners 1940A for the left leg and 1940B for the right leg, and tension members for the left leg and for the right leg in the form of compound cables 1930A and 1930B arranged to move in conduits 1928A and 1928B respectively, conduits 1928A and 1928B being in part located within or on inextensible material webs. Cables 1930A and 1930B may each comprise of a plurality of strands of substantially inextensible material, for example without limitation, PTFE, stainless steel, Nylon®, Kevlar®, one or more ultra-high molecular weight polyethylene-based fiber, and a fiber with a diamond weave. The fiber with a diamond weave may be, for example without limitation, cotton, polyester, polypropylene, and Technora®. Broken rectangle 100 indicates the region of the left leg shown in FIG. 1. In this embodiment, tensioned compound cables 1930A and 1930B are the mechanisms for generating the required force on the knee joints while the webs of inextensible material either serve as anchors to the torso or thigh, or they translate to the knee joint the forces generated by the tension in the cables. In order to perform its function, the strands of the cable are substantially inextensible, rendering compound cables 1930A and 1930B inextensible.

In FIG. 14A and FIG. 14B, focusing for now on applying pressure to the left knee, four strands of inextensible compound cable 1930A are laterally spread with respect to one another as they progress through left leg calf grip 1926A. Cable 1930A otherwise extends inside conduit 1928A. Inextensible web 1924A, which applies pressure to the medial side of the left knee, is attached at its two horizontal ends to conduit 1928A. When the tension in cable 1930A is increased, the pressure on the medial side of the left knee is commensurately increased via web 1924A due to the two portions of conduit 1928A being pulled away from each other.

In one embodiment, shown in FIG. 14A and FIG. 14B, conduit 1928A routes cable 1930A around the waist within belt 1910. Upper inextensible material web 1922A (see FIG. 14B) assures the positioning of conduit 1928A. Tensioner 1940A is disposed within the line of cable 1930A in order to adjust the tension in cable 1930A. Tensioner 1940A thereby controls pressure on the left knee and, in this embodiment, is disposed on the right hip. In other embodiments, tensioner 1940A may be disposed elsewhere along the route of conduit 1928A in the line of cable 1930A. Tension evener 1942A, described in more detail below, may be disposed somewhere along the route of conduit 1928A in the line of cable 1930A so that tension applied by tensioner 1940A is transmitted via tension evener 1942A to all the strands of compound cable 1930A. This arrangement places tension evener 1942A tension-wise in communication with tensioner 1940A and with the strands of tension member 1930A. In the examples to follow, we shall depict tension evener 1942A as disposed immediately adjacent to tensioner 1940A. In general, it may be located remote from tensioner 1940A, but tension-wise in communication with tensioner 1940A. The role of tension evener 1942A is to ensure that every strand of compound cable 1930A experiences the same tension despite the different individual paths followed by the different strands, for example around the triceps surae. How this tension evening is achieved will be described below in more detail.

Being directed to applying a suitable force to medial collateral ligament (MCL) 170 of FIG. 1, inextensible web 1924A of FIG. 14A is disposed to apply a force on the medial side of joint 100, the force being directed generally toward the lateral side of the left knee. This stabilizes the left knee joint against medial displacement as result of problems with medial collateral ligament 170 of the left knee. Correspondingly, inextensible web 1924B is disposed to apply a force on the medial side of the right knee, the force being directed generally toward the lateral side of the right knee. This stabilizes the right knee joint against medial displacement as result of problems with the medial collateral ligament of the right knee. Compound cable 1930A exits conduit 1928A to round in part the triceps surae in individual conduits routed through calf grip 1926A. Having rounded in part the triceps surae, the separate strands of compound cable 1930A resume their collective path through conduit 1928A.

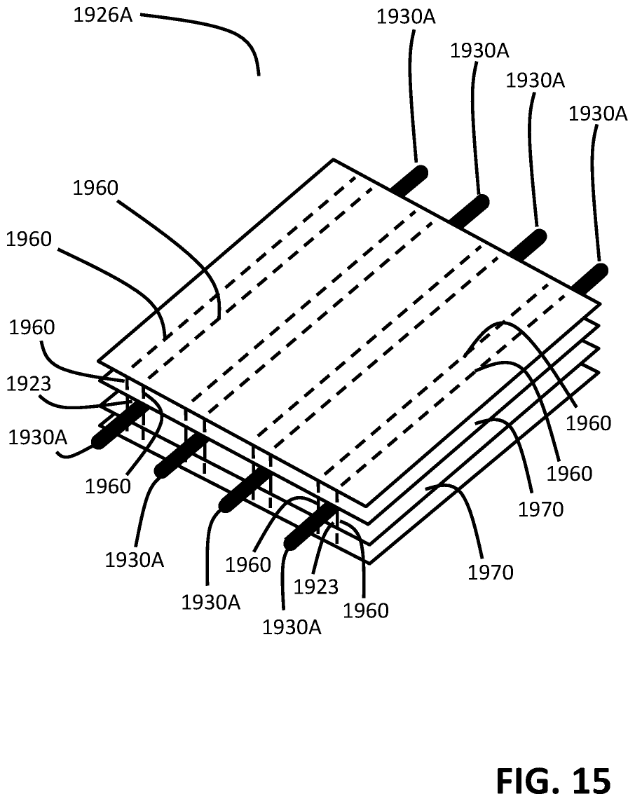

We turn now to tension evener 1942A and describe its working with reference to FIGS. 14A, 14B, 15 and 16A. In FIG. 15, the basic conduit arrangement of FIG. 6B is employed in routing the four strands of compound cable 1930A of FIGS. 14A and 14B through calf grip 1926A. In other embodiments, different numbers of strands may be employed in compound cable 1930A. Conduits 1923 within calf grip 1926A are defined by rows of stitching 1960 defining the lateral limits of a conduit and two layers of low friction material strips 1970 defining the inner and outer limits perpendicular to the skin of the user. Fabric 1970 of calf grip 1926A may comprise two layers, as shown in FIG. 15, or may in some embodiments comprise a single layer. In FIG. 15, the stitching is shown as parallel. In general, conduits 1923 are not limited to being parallel and may follow diverging and converging paths through fabric 1970 of calf grip 1926A. Their paths may be chosen to improve a grip of calf grip 1926A on the triceps surae. In further embodiments, the conduits employed may be of the type shown in FIG. 6A and FIG. 6C.

Fabric 1970 of calf grip 1926A may be the same as the general fabric of the garment. In other embodiments, fabric 1970 of calf grip 1926A may differ from the general fabric of brace garment 1900. The arrangement shown in FIGS. 14A, 14B, 15 and 16A has a first end of compound cable 1930A approaching tensioner 1940A clockwise around the waist on belt 1910 (looking down toward the feet) and a second end of compound cable 1930A approaching tensioner 1940A anti-clockwise around the waist on belt 1910. The first end of compound cable 1930A therefore approaches tensioner 1940A from the right of the drawing in FIG. 14A while the second end approaches tensioner 1940A from the left in the drawing of FIG. 14A.

FIG. 14B shows some aspects of the right leg of garment 1900 more clearly. It employs the same arrangement as the left leg described above. The elements of the right are differentiated from those pertaining to the left leg by the simple expedient of being labeled with a “B” rather than an “A” after the relevant numeral. Tensioner 1940B and tension evener 1942B for the right leg of garment 1900 are disposed on the left hip of the user in FIG. 14B.

In one embodiment, the second end of compound cable 1930A approaching tensioner 1940A in anti-clockwise fashion (from the left in the drawing of FIG. 14A) may be simply fixed in stationary configuration, either directly or indirectly, to tensioner 1940A. In this embodiment, all four anti-clockwise approaching strands of the second end of compound cable 1930A are fixed to tensioner 1940A.

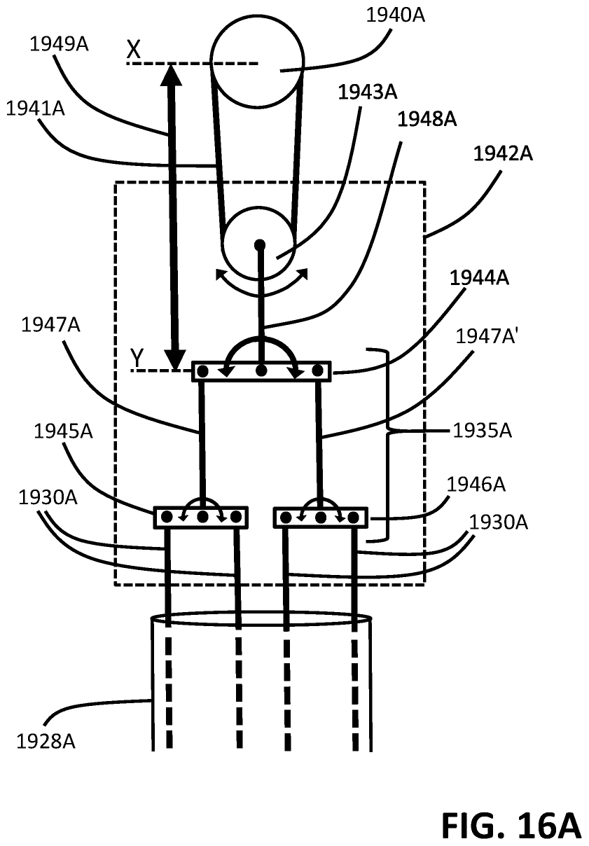

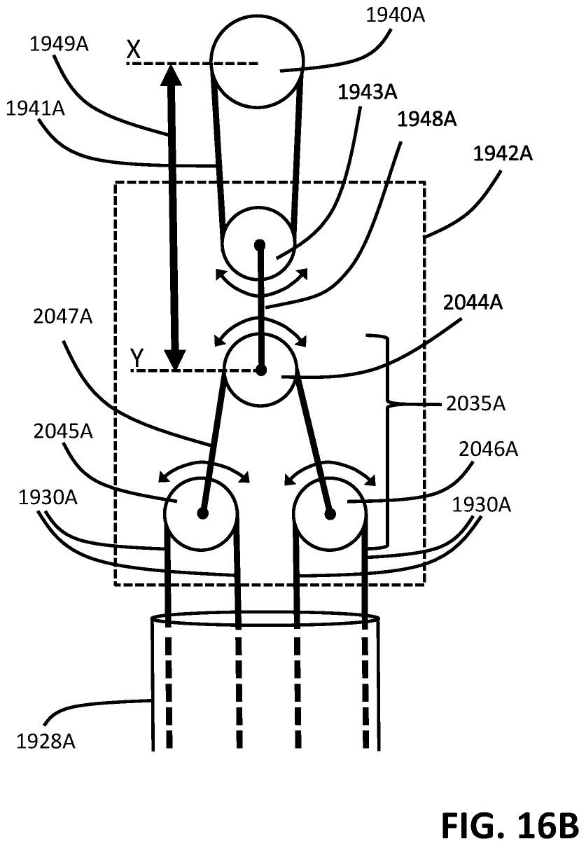

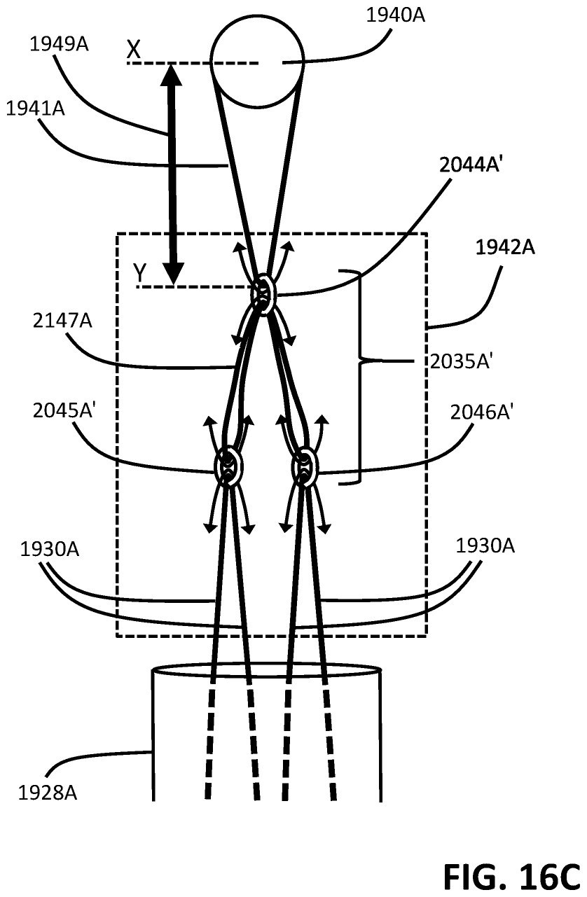

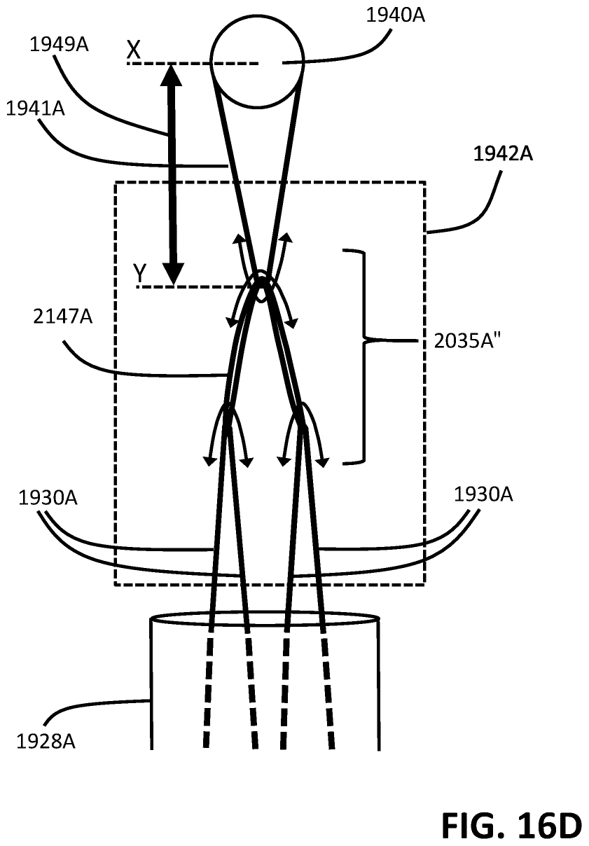

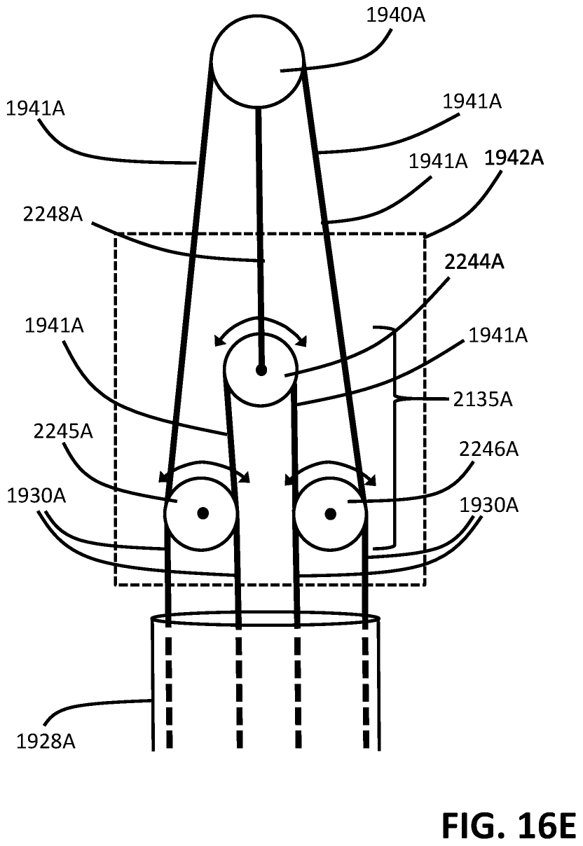

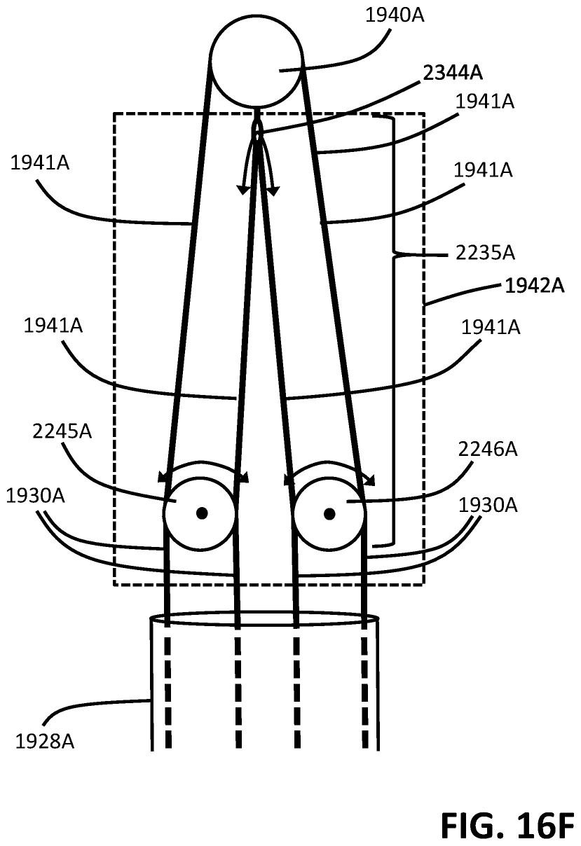

We turn now to the four strands of the first end of compound cable 1930A approaching tensioner 1940A from the right of the drawing in FIG. 14A and which are routed through calf grip 1926A and thence to tension evener 1942A. In embodiments shown in FIGS. 16A to 16E, tensioner 1940A is disposed to move elements of tension evener 1942 (for example, pulley 1943A of FIGS. 16A and 16B) and any elements attached to them along the direction indicated by bidirectional arrow 1949A. This is achieved by retracting or extending inextensible cable 1941A. To this end, one of two ends of inextensible cable 1941A may be fixedly attached to tensioner 1940A while the other end is retracted or extended by operating tensioner 1940A. In other embodiments, both ends of inextensible cable 1941A may be retracted or extended by operating tensioner 1940A.

FIG. 16A shows one embodiment of tension evener 1942A in more detail. Tension evener 1942A comprises a whippletree 1935A, a device well known from the era of the horse-drawn cart and the subject of several patents granted by the United States Patent and Trademark Office. One of its most common uses in the 21′ century is in evening out pressure on automobile windscreen wipers. Inextensible cable 1948A joins pulley 1943A to a fulcrum or pivot point of primary pivoting bar 1944A of whippletree 1935A. The term “whipple bar” is used herein to describe a pivoting bar of a whipple tree. The term “swingle” has also been used in some quarters to describe a pivoting bar of the type employed in a whipple tree. In other embodiments, inextensible cable 1941A may be engaged directly with whippletree 1935A, this being an embodiment in which pulley 1943A is obviated. Whippletree 1935A is described in more detail below. Various arrangements of pulleys and inextensible cables may be employed to move whippletree 1935A closer to and further away from tensioner 1940A.

Whippletree 1935A, as shown in the particular embodiment of FIG. 16A, is a “doubletree” whippletree, being comprised of two tiers of whipple bars, being bar 1944A in a first tier, and bars 1945A and 1946A in the second tier, in order to accommodate the four strands of compound cable 1930A described in FIG. 14A and FIG. 15. In FIG. 16A, the four strands of compound cable 1930A are shown connected to the second tier of whipple bars in FIG. 16A. In a more general embodiment, whippletree 1935A may have further tiers of whipple bars and engage more strands of a compound cable. The term “multi-tier whippletree” is used herein to describe a whippletree having a plurality of tiers of whipple bars.

Whipple bar 1944A is free to rotate about its fulcrum or pivot point as shown by the rotary arrow associated with whipple bar 1944A. If the tension in cable 1947A′ should be higher than the tension in cable 1947A, whipple bar 1946A will exert more force on pull harder than whipple bar 1945A on whipple bar 1944A, and whipple bar 1944A will pivot clockwise about its pivot point to a degree of rotation at which the difference in tension is evened out. The same action takes place if the two strands 1930A attached to whipple bar 1945A are under different amounts of tension. Whipple bar 1945A would pivot about its indicated pivot point to a degree of rotation at which the tension in the two strands attached to whipple bar 1945A are equal. The same behavior also holds for whipple bar 1946A in respect of any difference in tension between the two strands 1930A attached to it.

In FIGS. 14A, 14B, 15 and 16A a total of four strands are shown for compound cable 1930A. In general, any integer number N>1 of strands may be connected to a whippletree evener 1942A to balance tension by this general means. To employ three strands 1930A, two strands 1930A may be attached to whipple bar 1945A, while whipple bar 1946A may be omitted and the third strand 1930A attached directly to whipple bar 1944A. To employ more than four strands, an additional tier of whipple bars may be disposed between strands 1930A and whipple bars 1945A and 1946A of FIG. 16A, resulting in a “tripletree” whippletree. There is no conceptual limit to the number of strands of compound cable 1930A that may have their tension balanced in this way by a generalized whippletree evener.

In FIG. 16A, lever-and-fulcrum-based whippletree 1935A is employed to describe the concept, but in other embodiments of tension evener 1942A arrangements of other devices allowing a strand to move or slide over or through them may be employed, including devices with holes through which the strands may pass. Suitable devices of this type include without limitation pulleys, threadable beads, rings, eyelets, blocks, loops of inextensible fabric, and loops in other cables or strands. The term “block” is used herein to specifically describe a single- or double-holed block as used in a “block and tackle”. The devices may have a connecting mechanism that allows them to be engaged with another functionally similar device. The mechanism to connect them to another functionally similar device may be a substantially inextensible cable, or thread, or strand. In some embodiments the connection mechanism may be a cable of the same material as compound cable 1930A or a strand of cable 1930A.

While calf grip 1926A may be made from an extensible material, including for example rubber, to thereby avoid employing laterally spread strands of compound cable 1930A and tension 1942A evener, such an attempted solution for suitable grip behavior does not overcome the problem of that extensible material having to be customized for wearers of brace garment 1900. The use of the laterally spread strands of compound cable 1930A and tension 1942A evener overcomes that limitation.

In FIG. 16B we turn to an alternative embodiment of the concept of tension evener 1942A of FIG. 16A. In FIG. 16B tensioner 1940A is disposed to move the assemblage of pulleys 1943A, 1944A, 1945A and 1946A along the direction indicated by bidirectional arrow 1949A. This is achieved by retracting or extending inextensible cable 1941A. To this end, one of two ends of inextensible cable 1941A may be fixedly attached to tensioner 1940A while the other end is retracted or extended by operating tensioner 1940A. In other embodiments, both ends of inextensible cable 1941A may be retracted or extended by operating tensioner 1940A. Inextensible cable 1948A joins pulley 1943A to pulley 2044A. In other embodiments inextensible cable 1941A may be engaged directly with pulley 2044A to slide over a portion of pulley 2044A, this being an embodiment in which pulley 1943A is obviated. Various arrangements of pulleys and inextensible cables may be employed to move pulley 2044A closer to and further away from tensioner 1940A.

The four strands of inextensible compound cable 1930A are shown in FIG. 16B as being in fact two looped strands, each folded back upon itself, one strand looped about pulley 2045A and the other strand looped about pulley 2046A. Both ends of each of the two strands is fastened to tensioner 1940A when the strands reach tensioner 1940A, having navigated the entire predetermined path of compound cable 1930A about the left leg and body of the user and having approached tensioner 1940A in the clockwise direction as already explained above. Pulleys 2045A and 2046A are connected by inextensible cable 2047A engaged with pulley 2044A. If the tension in strand 1930A folded about pulley 2045A is not equal in its two portions either side of pulley 2045A, then that strand 1930A will move about pulley 2045A, either by sliding or by rotating pulley 2045A, until that tension is even. The same is true for strand 1930A folded about pulley 2046A.

When the resolved tension in strand 1930A about pulley 2045A and the resolved tension in strand 1930A about pulley 2046A are mutually unbalanced, the two pulleys will move with respect to each other as cable 2047A slides over pulley 2044A or rotates pulley 2044A to even out the tension in strands 1930A. This tension evening arrangement functions like whippletree 1935 of FIG. 16A. The degrees of rotation of the various pulleys are determined by the tension differences already described and the radii of the pulleys. The difference in the embodiment of FIG. 16B is that whipple bars 1944A, 1945A and 1946A of FIG. 16A are replaced here by pulleys 2044A, 2045A and 2046A, while cables 1947A and 1947A′ are replaced by single cable 2047A. The particular embodiment in FIG. 16B is also a “doubletree” (two-tier) whippletree 2035A. As a parallel to the arrangement of FIG. 16A, the embodiment of FIG. 16B may be similarly extended to encompass more tiers of pulleys and larger or odd numbers of strands. In general, the whippletree embodiment in FIG. 16B is another example of a multi-tier whippletree.

A further embodiment of tension evener 1942A is shown in FIG. 16C in which the pulleys of FIG. 16B are replaced by rings through which cables and strands are looped. The rings may be of any suitable material capable of withstanding the applied tension, including but not limited to metal rings, plastic rings and fabric rings or loops of fabric. More specifically, cable 2047A of FIG. 16B is replaced by looped inextensible cable 2147A. As in FIG. 16B, two strands 1930A looped back on themselves are employed and are threaded through rings 2045A′ and 2046A′ disposed at two ends of inextensible cable 2147A, thereby obviating pulleys 2045A and 2046A. Inextensible cable 1941A applying tension from tensioner 1940A is threaded directly through ring 2044A′ and cable 2147A is threaded directly through ring 2044A′. Pulley 1943A of FIG. 16B is thereby obviated as is cable 1948A of FIG. 16B. In this embodiment, tension evener 1942A and whippletree 2035A′ comprise no pulleys or whipple bars and the working of the device is based exclusively on cables and strands sliding within rings of the types described above. The functioning is nevertheless the same as in FIG. 16B. As a parallel to the arrangements of FIG. 16A and FIG. 16B, the embodiment of FIG. 16C may be similarly extended to encompass more tiers of rings and larger or odd numbers of strands. In general, the ring-based whippletree embodiment in FIG. 16C is another example of a multi-tier whippletree.

A further embodiment of tension evener 1942A, derived from the arrangement in FIG. 16C, is shown in FIG. 16D in which the rings of FIG. 16C are replaced by loops in cables and strands. As in FIG. 16C, two strands 1930A looped back on themselves are employed and are threaded through the ends of inextensible cable 2147A, thereby obviating rings 2045A′ and 2046A′. Cable 2147A is threaded directly through cable 1941A. In this embodiment, tension evener 1942A and whippletree 2035A″ comprise no rings, pulleys, or whipple bars and the working of the device is based exclusively on cables and strands sliding within loops of other strands or cables. The functioning is nevertheless the same as in FIG. 16C. As a parallel to the arrangements of FIG. 16A, FIG. 16B, and FIG. 16C, the embodiment of FIG. 16D may be similarly extended to encompass more tiers of cable loops and larger or odd numbers of strands. In general, the loop-based whippletree embodiment in FIG. 16D is another example of a multi-tier whippletree.

Alternative embodiments of tension evener 1942A and its interaction with tensioner 1940A include the example shown in FIG. 16E. For the sake of clarity, we show this embodiment using pulleys as in FIG. 16B. In this embodiment inextensible cable 1941A is also extended and retracted by tensioner 1940A to adjust tension in the system, but is engaged with both pulley 2245A and pulley 2246A. Between engaging with pulley 2245A and pulley 2246A, cable 1941A also engages with pulley 2244A. Cable 2248A serves simply to fixedly attach pulley 2244A to tensioner 1940A. As in FIG. 16B, cable strands 1930A are also engaged with pulleys 2245A and 2246A. Comparison with FIGS. 16B and 16C shows that this arrangement is equivalent to omitting respectively cable 2047A and cable 2147A and assigning their tension evening function to cable 1941A in whippletree 2135A. The motion of cable 1941A and strands 1930A in this embodiment may be a combination of sliding over and rotation of the pulleys they are engaged with. This arrangement remains a two-tier whippletree and is therefore also an example of a multi-tier whippletree. As a parallel to the arrangements of FIGS. 16A, 16B, 16C, and 16D, the embodiment of FIG. 16E may be similarly extended to encompass more tiers of pulleys and larger or odd numbers of strands.

In FIG. 16F, a variant of the embodiment in FIG. 16E is shown in which cable 2248A and pulley 2244A of FIG. 16E are simply replaced with eyelet 2344A, a label specifically chosen to indicate that eyelet 2344A fulfills the same role in FIG. 16E as pulley 2244A does in FIG. 16E. Eyelet 2344A may be made from any material that can withstand the tension applied to it. As a parallel to the arrangements of FIGS. 16A, 16B, 16C, 16D, and 16E, the embodiment of FIG. 16F may be similarly extended to encompass more tiers of pulleys and larger or odd numbers of strands. For example, a further eyelet 2344A may be added to tensioner 1940 and cable 1941A may engage with a third pulley disposed between pulleys 2245A and 2246A, the third pulley engaging with another strand 1930A, thereby allowing six strands (that is, two more double-strands) to be threaded through calf grip 1926A of FIGS. 14A and 14B. The arrangement 2235A in FIG. 16F, and that of its extension described here, remain two-tier whippletrees and are therefore also examples of multi-tier whippletrees. As shown in FIG. 16D with respect to FIGS. 16A, 16B, and 16C, the pulleys of FIGS. 16E and 16F may be replaced by suitably arranged cable loops. Some embodiments, for example the embodiment shown in FIG. 16E, may have mechanical advantage in that the force exerted on cable 1941A at tensioner 1940A results in a greater force on cable strands 1930A.

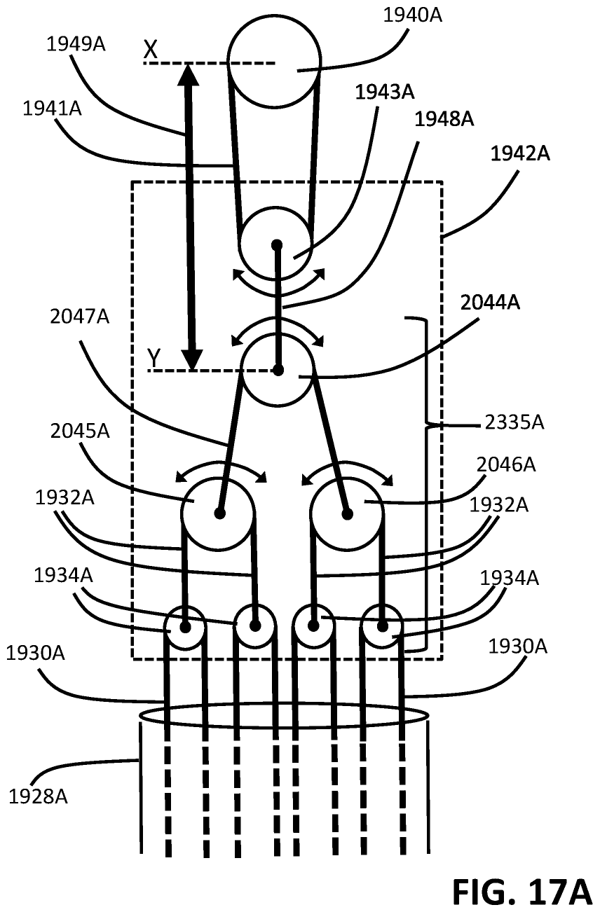

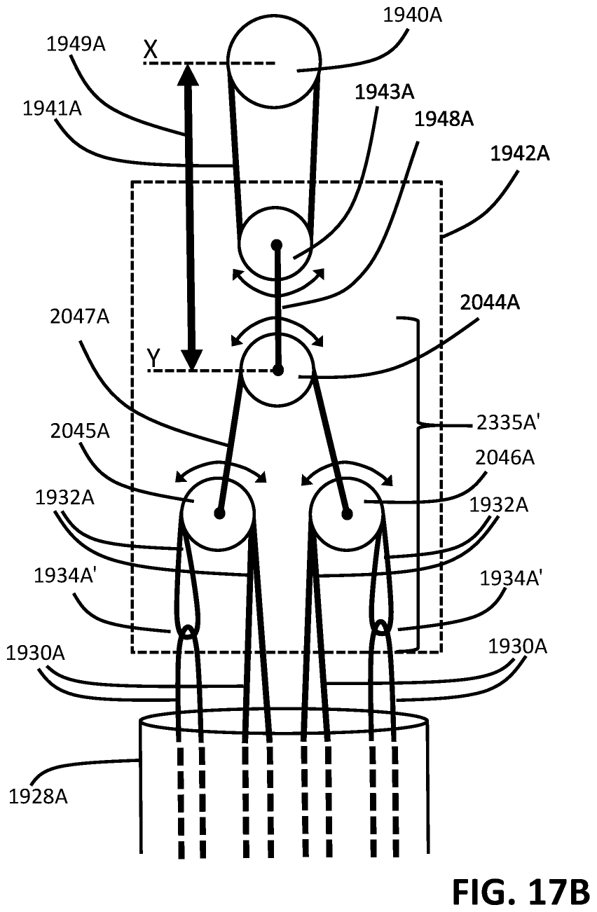

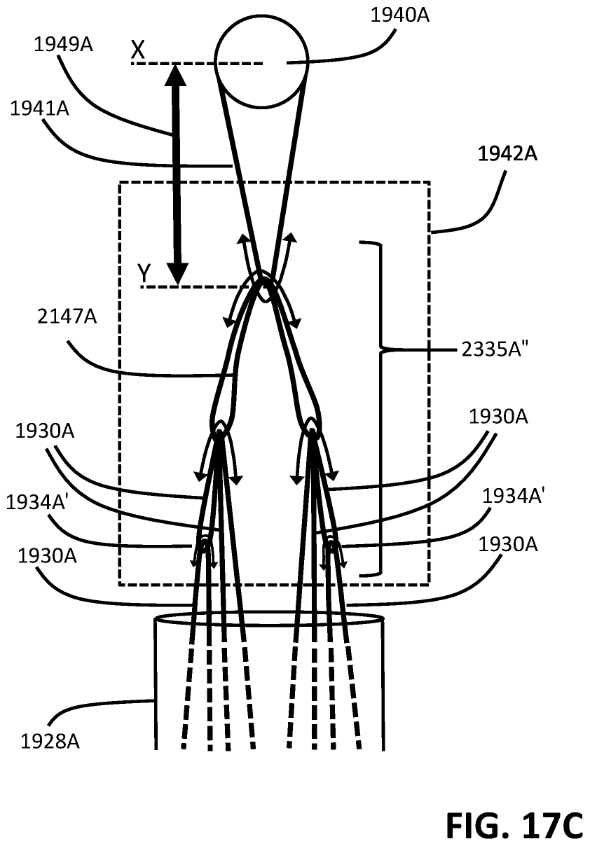

FIG. 17A shows how alternative tension evener 1942A comprising three-tier whippletree 2335A may be employed to even the tension in eight strands. Eight strands 1930A arise from four strands being looped over pulleys at the one end of compound cable 1930 and then proceed via conduit 1928A to be threaded individually through calf grip 1926A of FIGS. 14A and 14B. They then subsequently proceed as compound cable 1930A via conduit 1928A to engage at the other end of cable 1930A with tensioner 1940A in the different extendable and fixed ways already described above. The three-tier whippletree may be realized in any of the forms described with reference to FIGS. 16A to 16F. For the sake of clarity, a pulley-based implementation, building on the system of FIG. 16B, is chosen. In this embodiment, two inextensible cables 1932A are fixed at their four ends to four pulleys 1934A. The eight strands 1930A are engaged with pulleys 1934A. The tension evening function of this embodiment proceeds as already explained, except that there is in FIG. 17A an additional tier of pulleys 1934A and cables 1932A to undertake the balancing of the tension in strands 1930A. In all other respects, the embodiment of FIG. 17A is the same as that of FIG. 16B and the remaining elements bear the same labels as in FIG. 16B.

FIG. 17B shows yet another embodiment of three tier whippletree arrangement 2335A′ based on the whippletree arrangement of FIG. 17A. In the embodiment of FIG. 17B, pulleys 1934A are absent. Instead, two of four pulleys 1934 are functionally replaced by mutual looping 1934A′ of pairs of continuous strands 1930A, while strands 1930A are routed over pulleys 2045A and 2046A. This remains a three-tier whippletree, though the third tier, unlike that of FIG. 17A, has only two pivot points. To convert the embodiment of FIG. 17B into a whippletree evener of which the third tier would have four pivot points, the same arrangement maybe employed about each of pulleys 2045A and 2046A as is used for cable 2147A of FIG. 16C. In such an arrangement, none of cables 1930A would be engaged with a pulley and each would have a looping arrangement 1934A′, for a total of four.

FIG. 17C shows yet a further embodiment of three-tier whippletree arrangement 2335″ based on the whippletree arrangement of FIG. 16D and employing looped strand 1930A configuration of FIG. 17B and loopings 1934A′. We employ the labeling of FIG. 16D for the rest of the structure as it is identical to that embodiment.

As already explained for the two-tier whippletree with reference to FIGS. 16B to 16F, the three-tier arrangements may also be implemented in other mechanical variants, including rings, eyelets or blocks of suitable materials to withstand the tension, including fabric, in order to serve the same function. The embodiments of FIGS. 17A, 17B and 17C also rely on a multi-tier whippletree tension evener.

FIGS. 16A to 16F and FIGS. 17A to 17C all describe a multi-tier whippletree tension evener using the left leg arrangement of garment 1900 in FIGS. 14A and 14B. The same basic arrangement may be made for multi-tier whippletree tension eveners 1942B for use with the right leg tensioning. FIGS. 14A and 14B show inextensible webs 1924A and 1924B, disposed to apply pressure to the medial sides of the left knee and right knee respectively. Similar inextensible webs may be arranged, along with suitable tension members, conduits, inextensible cables, tensioners and tension eveners in order to apply pressure to the lateral sides of the two knees. The tensioner-evener pairs may be implemented according to FIGS. 16A to 16F and FIGS. 17A to 17C. In some garments there may be a need to implement only a subset of the tensioner-evener pairs according to FIGS. 16A to 16F and FIGS. 17A to 17C. Some embodiments of the garment may comprise both lateral and medial arrangements of inextensible webs, as per the foregoing, in order to simultaneously apply pressure to both the lateral and medial sides of one or both knees.

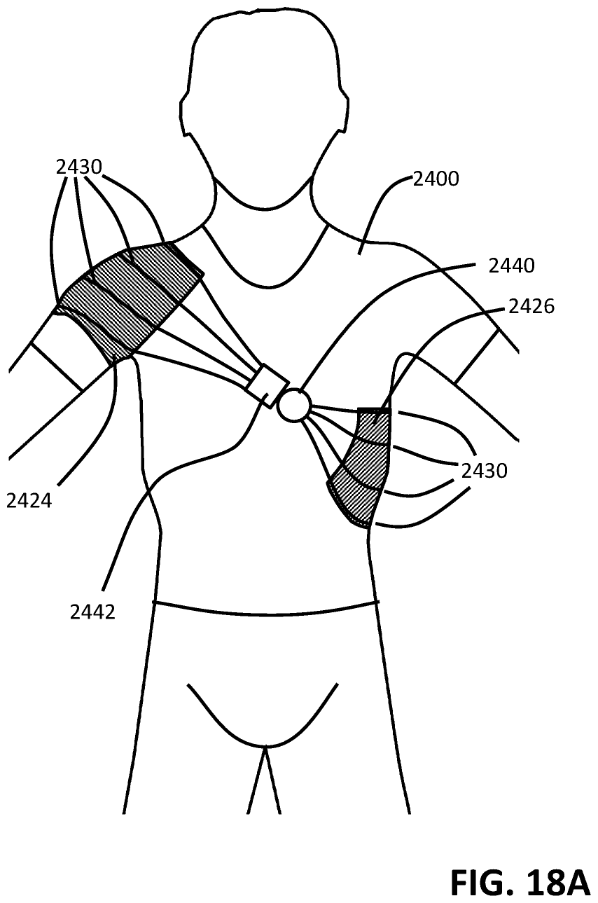

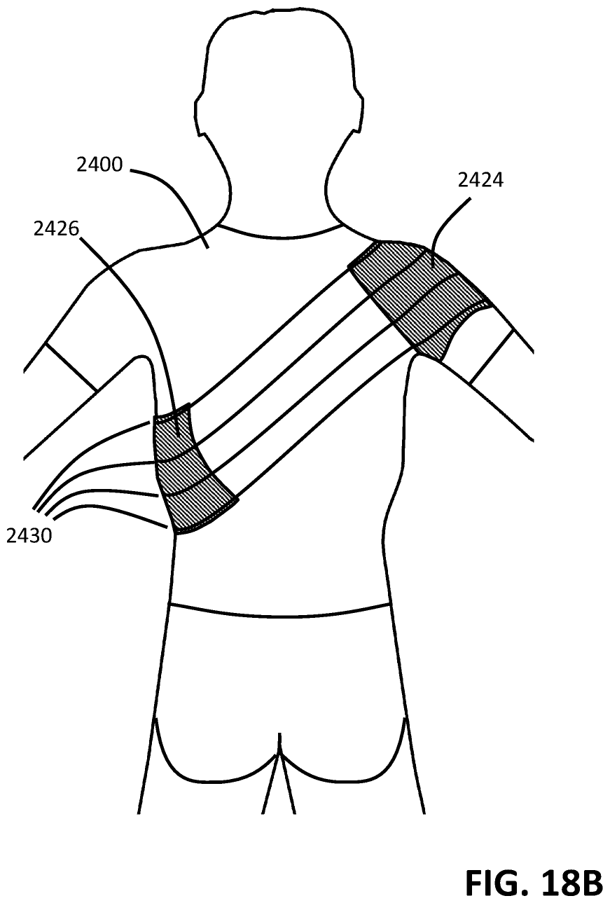

The example of a human knee has been used above to describe brace garments 200, 200″, and 1900 for stabilizing a human knee. The concepts described herein are, however, not limited to the human knee and may be applied to any anatomical joint to be stabilized, including for example without limitation, a human ankle, elbow, wrist, shoulder, hip, neck, spine, and one or more vertebrae. FIGS. 18A and 18B show an example of a right shoulder joint being stabilized by shoulder brace garment 2400 enveloping an upper portion of the torso of a wearer as well as adjoining portions of the upper arms, thereby enveloping the shoulders. Inextensible web 2424 provides pressure on a selected injured or unstable shoulder joint under the action of a tension member in the form of inextensible cable 2430 that follows a path around the torso in order to anchor grip the ribcage of the wearer on the side of the torso opposing the side of the torso on which inextensible web 2424 is disposed. As in FIGS. 14A and 14B, inextensible cable 2430 may be divided into a plurality of constituent strands laterally spread with respect to one another, four strands 2430 being used in FIGS. 18A and 18B, routed through grip 2426 within strand conduits already described with reference to FIGS. 14A and 14B. The fabric of grip 2426A may be the same as the general fabric of garment 2400. In other embodiments, the fabric of grip 2426 may differ from the general fabric of brace garment 2400. Tensioner 2440 may be the same as the tensioner in the embodiments shown in FIGS. 14A and 14B. Tension evener 2442 may be the same as the tensioners of FIGS. 16A to 16F, tension-wise in communication with the tensioner and with the strands of the tension member. As described with reference to the tension eveners of FIGS. 16A to 16F and FIGS. 17A to 17C, the number of strands may be any number equal to or greater than two and tension evener 2442 may comprise a whippletree which may be a multi-tier whippletree.

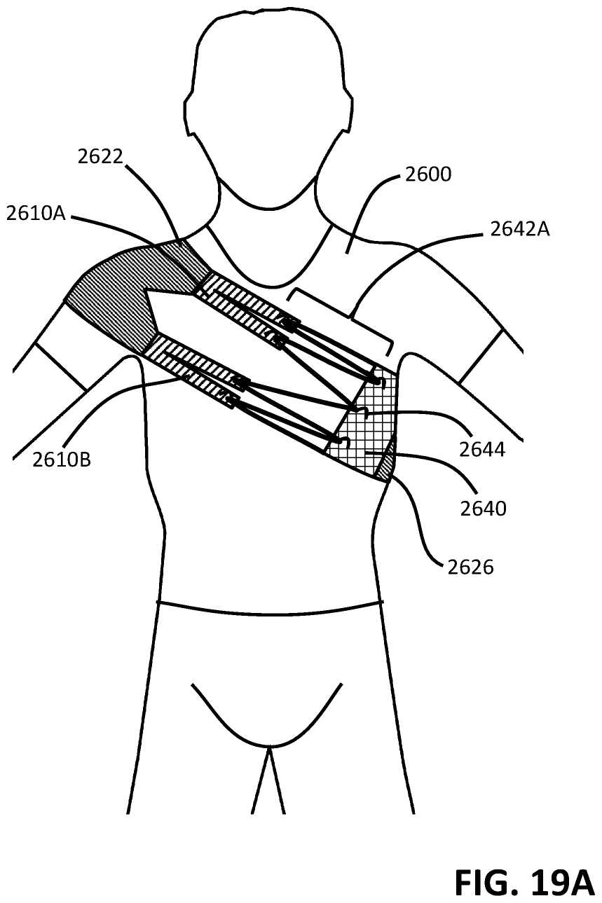

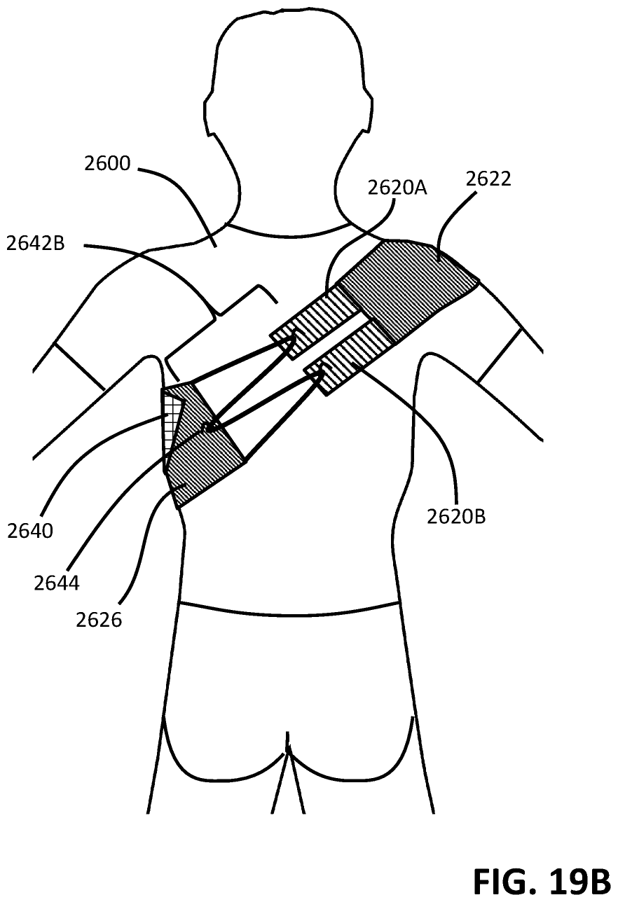

FIGS. 19A and 19B show another embodiment of a right shoulder joint being stabilized by shoulder brace garment 2600 enveloping an upper portion of the torso of a wearer as well as adjoining portions of the upper arms, thereby enveloping the shoulders. Shoulder portion 2622 of shoulder brace garment 2600 provides pressure on a selected injured or unstable shoulder joint under the action of a compound tension member that follows a path around the torso in order to anchor grip the ribcage of the wearer on the side of the torso opposing the side of the torso on which shoulder portion 2622 is disposed. In this embodiment, the tension member comprises shoulder portion 2622; straps 2610A, 2610B, 2620A, and 2620B; whippletree eveners 2642A and 2642B; and a tensioner comprising two mutually engaging portions of hook-and-loop fabrics 2626 and 2640. Tension is applied by pulling portions 2626 and 2640 over each other in generally opposing directions and mutually engaging them. Whippletree eveners 2642A and 2642B balance the tension along the paths described by straps 2610A and 2620A on the one hand, and straps 2610B and 2620B on the other by substantially inextensible threads of the whippletrees moving through eyelets 2644 serving as threadable members. Garment 2600 is shown as enveloping the entire upper torso, but is not required to envelope the unbraced left shoulder. In principle, brace garment 2600 does not need to comprise a T-shirt as shown in FIGS. 19A and 19B and may comprise only the other components numbered hereabove. As will be noted, the embodiment in FIGS. 19A and 19B, unlike that of FIGS. 18A and 18B, does not require the use of any conduits for tension members, nor does it have a discrete localized tensioner or a discrete localized tension evener. The tensioning is distributed, as is the tension evening. In other embodiments, the hook-and-loop tensioner described by fabric portions 2626 and 2640 may be substituted for by other tensioning arrangements to engage with whippletree eveners 2642A and 2642B. The tensioner may be the same as the tensioner in the embodiments shown in FIGS. 14A and 14B. Whippletree eveners 2642A and 2642B may be substituted for by any of the whippletree eveners of FIGS. 16A to 16F, or by any tension eveners that can engage with the tensioner and with straps 2610A, 2610B, 2620A, and 2620B.

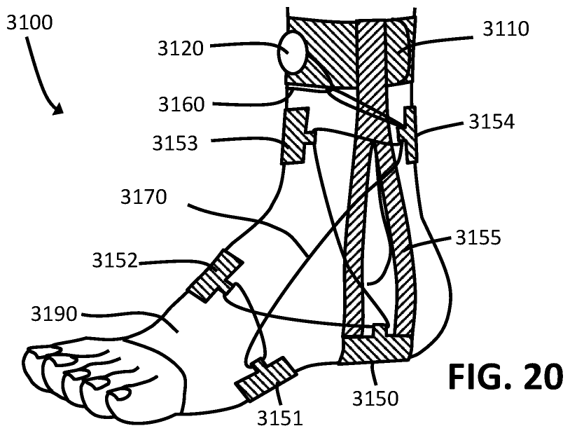

FIG. 20 shows wearable brace garment 3100 for a human ankle. In this example embodiment, a left foot is used as an example, although a mirror of the structure of wearable brace garment 3100 would be suitable for a right foot (not shown). The lateral side of the foot is shown, the medial side having the same arrangement, with inextensible webs 3150 and 3151 extending under the foot from the lateral to the medial side, inextensible web 3152 extending over the foot from the lateral to the medial side, inextensible web 3153 extending from the lateral to the medial side of the leg across the anterior of the lower leg, and inextensible web 3154 extending from the lateral to the medial side of the leg across the posterior of the lower leg. The tension member arrangement to be explained below is therefore substantially the same on the medial side of the leg as on the lateral side shown in FIG. 20.

In addition to inextensible webs 3150, 3151, 3152, 3153 and 3154 already described, inextensible web 3155 (and its equivalent on the medial side of the leg) may be integral to web 3150, or may be a separate inextensible web engaged with inextensible web 3150 by a hook-and-loop or other arrangement. Anchor band 3110 fits around the lower leg above the ankle and may be tightened around the leg by any of a number of arrangements, including the overlapping hook-and-loop arrangement shown in FIG. 20. Inextensible web 3155 (and its equivalent on the medial side of the leg) may attach to anchor band 3110 via a hook-and-loop arrangement or any other stable attachment mechanism.

To apply a suitable stabilizing force to the ankle joint, tensioner 3120 is provided on anchor band 3110. Tensioner 3120 applies the required force to inextensible webs 3150, 3151, 3152, 3153 and 3154 by means of two loops of substantially inextensible thread engaged in a whippletree arrangement with inextensible thread 3160. The loop on the lateral side of the ankle is labelled 3170. The loop on the medial side is not visible in FIG. 20, being obscured by the foot, but it follows a path that is substantially a mirror image of the path of thread 3170. When tensioner 3120 is tightened, substantially inextensible loop 3170 (and its equivalent on the medial side of the leg) slide through loop 3160 of the whippletree and through threadable members on inextensible webs 3150, 3151, 3152, 3153 and 3154 to balance the tension between loop 3170 and its equivalent on the medial side of the leg. This also balances the forces applied between the lateral and medial sides of the ankle joint. Wearable brace garment 3100 may comprise sock 3190 to which the foregoing elements are attached. Sock 3190 may be a whole sock or a toeless sock.

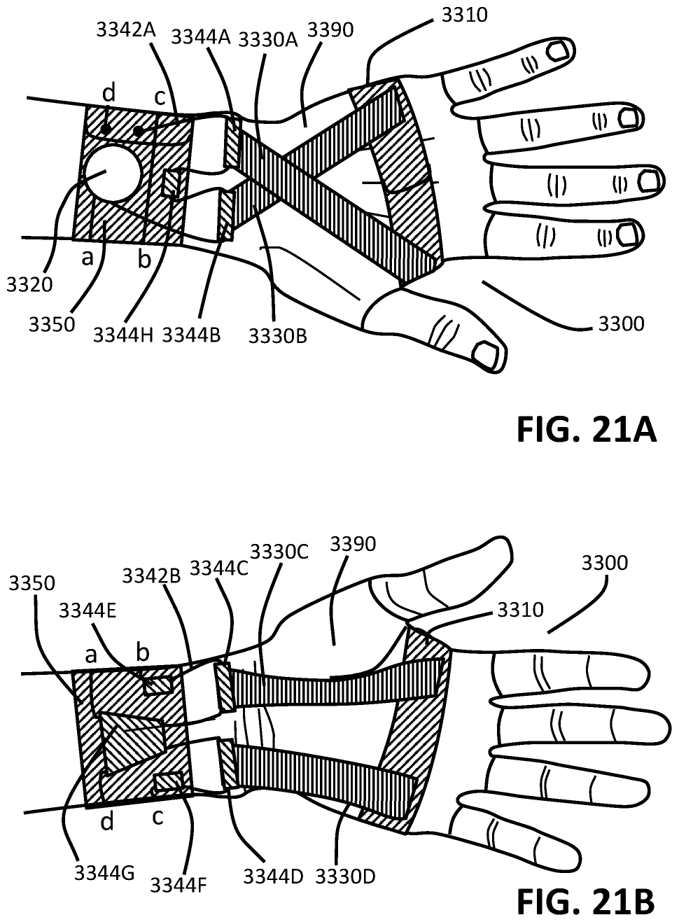

FIGS. 21A and 21B show wearable brace garment 3300 for a human wrist. Brace garment 3300 comprises hand anchor band 3310, fastened around the hand by a hook-and-loop or other releasable fastening arrangement, and arm anchor band 3350, fastened around the forearm by means of a hook-and-loop or other releasable fastening arrangement. Dorsal straps 3330A and 3330B are attached at their distal ends to hand anchor band 3310 on the dorsal side of the hand. The attachment may be permanent or may be removable via a hook-and-loop type fastening. Dorsal straps 3330A and 3330B may be inextensible. Palmar straps 3330C and 3330D are attached at their distal ends to hand anchor band 3310 on the palmar side of the hand. The attachment may be permanent or may be removable via a hook-and-loop type fastening. Palmar straps 3330C and 3330D may be inextensible. All four straps 3330A, 3330B, 3330C and 3330D may have respective loops or conduits 3344A, 3344B, 3344C, 3344D at their proximal ends.

Tensioner 3320 is disposed on the dorsal side of arm anchor band 3350 and engages with straps 3330A, 3330B, 3330C and 3330D by means of substantially inextensible cables or threads 3342A and 3342B. Cable 3342A passes through loops or threadable members 3344A and 3344B, as well as loop or threadable member 3344H disposed on the dorsal side of arm anchor band 3350. One end of cable 3342A is tied to arm anchor band 3350. The other end of cable 3342A may be tied to tensioner 3320. As tension is increased in cable 3342A by operating tensioner 3320, whippletree tension evener formed by cable 3342A and threadable members 3344A, 3344B and 3344H ensures that the tension in dorsal strap 3330A and the tension in dorsal strap 3330B are the same.

One end of cable 3342B may be tied to arm anchor band 3350 and the other to tensioner 3320 to allow tensioner 3320 to adjust the tension in cable 3342B. The whippletree evener formed by cable 3342B in combination with threadable members 3344C, 3344D, 3344E, 3344F and 3344G on the palmar side of brace garment 3300 ensures that the tension in palmar strap 3330C and the tension in palmar strap 3330D are the same. Labels a, b, c and d are aides in tracing the route of cable 3342 proximate the wrist around the two sides of anchor band 3350 shown in FIGS. 21A and 21B. Adjustment of tensioner 2230 applies a force to the wrist in order to stabilize it. Brace garment 3300 may comprise glove 3310 to which the arrangement described above may be attached. Glove 3310 may be a full glove or it may be a fingerless glove.

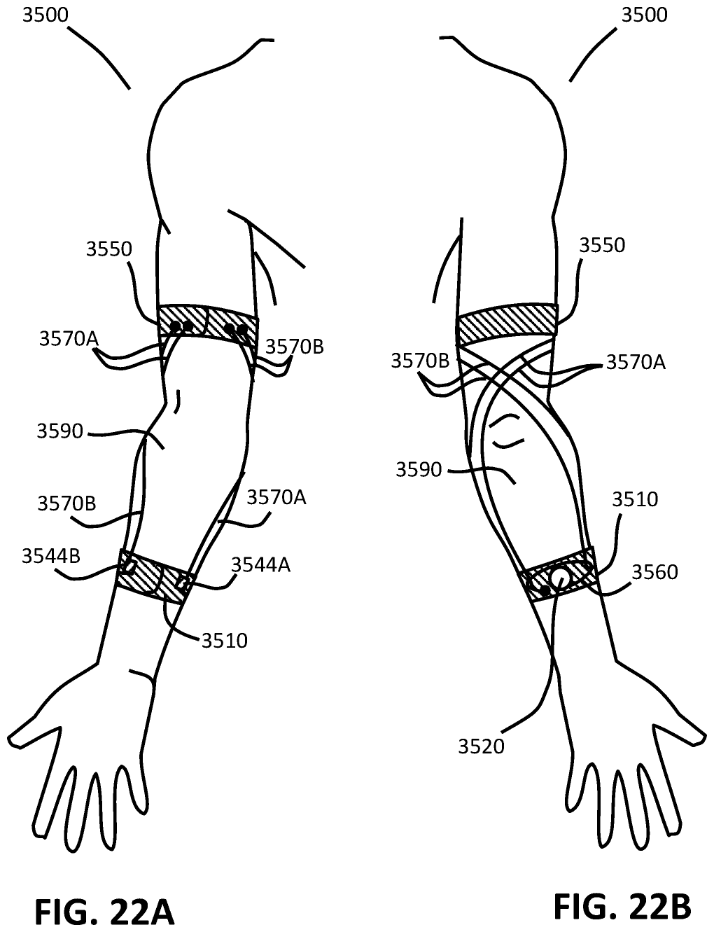

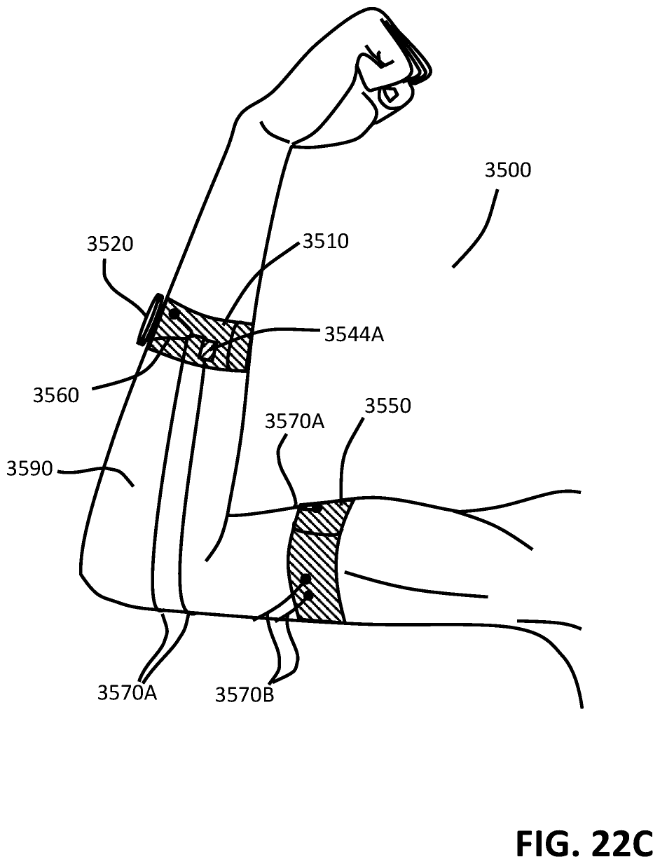

FIGS. 22A, 22B and 22C show wearable brace garment 3500 for stabilizing a human elbow. In FIGS. 22A, 22B and 22C brace garment 3500 is shown in different aspect views as applied to the right elbow of a human wearer. FIG. 22A shows an anterior view of the right elbow with palmar view of the right hand, while FIG. 22B shows a posterior view of the same elbow with a dorsal view of the right hand. FIG. 22C shows the same right arm and right hand in a third perspective view with the biceps contracted. Forearm anchor band 3510 and upper arm anchor band 3550 are releasably fastened to the forearm and upper arm respectively. To this end, both anchor bands may be equipped with hook-and-loop fastening facilities or other arrangements facilitating ease of attachment to the two parts of the arm. As shown in FIGS. 22B and 22C, tensioner 3520 is disposed on the dorsal side of forearm anchor band 3510. One end of substantially inextensible cable 3560 is attached to forearm anchor band 3510 and the other end of cable 3560 engages with tensioner 3520 at its other end, allowing tensioner 3520 to adjust a tension in cable 3560. Substantially inextensible cables 3570A and 3570B both have both of their ends attached to upper arm anchor band 3550. Cables 3570A and 3570B and are both looped through cable 3560. In order to guide cable 3570A it is additionally looped through threadable member 3544A on forearm anchor band 3510. In order to guide cable 3570B it is additionally looped through threadable member 3544B on forearm anchor band 3510.

Cables 3570A and 3570B may be routed along conduits extending between forearm anchor band 3510 and upper arm anchor band 3550. In some embodiments, brace garment 3500 may further comprise tubular garment 3590 extending from a proximal end at upper arm anchor band 3550 to a distal end at forearm anchor band 3510. The conduits for cables 3570A and 3570B may be disposed on or within tubular garment 3590. The conduits may be implemented according to the arrangements described at the hand of FIGS. 6A, 6b or 6C.

Cables 3570A and 3570B, together with cable 3560 and threadable members 3544A and 3544B, constitute a whippletree evener which serves to equate the tension in cables 3570A and 3570B. Adjusting tensioner 3520 causes the tension in cables 3570A and 3570B to adjust and the whippletree evener automatically ensures that the tension in each of the two cables 3570A and 3570B are the same. The tension in cables 3570A and 3570B produces the force required to stabilize the elbow joint of the user.

FIGS. 14A to 22C describe aspects and embodiments of a wearable brace garment 1900, 2400, 2600, 3100, 3300, 3500 for stabilizing an articulating joint of a wearer, the garment arranged for enveloping at least in part the joint. The garment may comprise a tension member disposed along a three-dimensional spatial path about the joint. The tension member varies among embodiments, but may comprise a plurality of strands substantially inextensible along a longitudinal direction. The brace garment may further comprise a tensioner disposed on the garment and arranged for adjusting a tension in the tension member. The tensioner may vary among embodiments but is exemplified by tensioners 1940A, 1940B, 2440, 3120, 3320 and 3520, as well as the hook-an-loop arrangement of FIGS. 19A and 19B. The brace garment may further comprise a tension evener 1942A, 1942B, 2442, 2642A, 2642B (as well as the cable arrangements of FIGS. 20 to 22C), disposed on the garment tension-wise in communication with the tensioner and with the strands of the tension member, the tension evener arranged for evening tension among the plurality of inextensible strands of the tension member.

The tension evener may comprise a whippletree, which may be a multi-tier whippletree. Suitable whippletrees for use as tension eveners for various of the embodiments are shown in FIGS. 12 and 13, as well as in FIGS. 16A to 17C. The whippletree may comprise pulleys (for example, 1943A, 2044A, 2045A, 2046A) disposed for engaging with the substantially inextensible strands of the tension member. The pulleys may made of one of wood, metal, and a polymer. In some embodiments, the whippletree may comprise one of injection-molded pulleys and three-dimensionally printed pulleys. The whippletree may comprise threadable members disposed for engaging with the substantially inextensible strands of the tension member. The threadable members may be beads, blocks, rings, eyelets and/or whipple bars disposed for engaging with the substantially inextensible strands of the tension member. The beads, blocks, rings, eyelets or whipple bars may be made of one of wood, metal, and a polymer. The threadable members may be injection-molded or three-dimensionally printed. The whippletree may comprise loops of substantially inextensible fabric disposed for engaging with the substantially inextensible strands of the tension member. The whippletree may be configured to provide a mechanical advantage.

The brace garment may comprise a web portion comprising the strands laterally spread, each strand disposed in a corresponding strand conduit on or within the garment. The garment may be made of a garment material and each conduit may have a lower coefficient of friction with respect to the strands than the coefficient of friction of the garment material with respect to the strands. Each conduit may comprise a tube of material different from the garment material. The three-dimensional spatial path of the tension member may extend around a limb articulating at the joint. The three-dimensional spatial path of the tension member may be arranged so that tension in the tension member produces a compressive force proximate the joint. The joint may be one of an ankle, elbow, wrist, shoulder, hip, neck, spine, and one or more vertebrae of the wearer. The tensioner may be configured to provide a mechanical advantage in applying tension to the tension evener.

FIGS. 14A and 14B, as well as FIGS. 18A and 18B describe a wearable brace garment 1900, 2400 for stabilizing an articulating joint of a wearer, garment 1900, 2400 arranged for enveloping at least in part the joint, the garment comprising: a tension member disposed along a three-dimensional spatial path about the joint in conduits on or within the garment 1900, 2400, the tension member comprising a plurality of strands 1930A, 1930B, 2430 substantially inextensible along a longitudinal direction; a web portion 1926A, 1926B, 2426 comprising the strands laterally spread, each strand disposed in a corresponding strand conduit; a tensioner 1940A, 1940B, 2440 disposed on the garment 1900, 2400 and arranged for adjusting a tension in the tension member; a tension evener 1942A, 1942B, 2442 disposed on the garment 1900, 2400 tension-wise in communication with the tensioner 1940A, 1940B, 2440 and with the strands 1930A, 1930B, 2430 of the tension member, the tension evener 1942A, 1942B, 2442 arranged for evening tension among the plurality of inextensible strands 1930A, 1930B, 2430 of the tension member.

The tension evener may comprise a whippletree 1935A, 2035A, 2035A′, 2035A″, 2135A, 2235A, 2335A, 2335A′, 2335A″ that may be a multi-tier whippletree. The whippletree may more particularly be a two-tier whippletree or a three-tier whippletree and the whippletree may employ one or more of pulleys and threadable members. The threadable members may include one or more of beads, rings, eyelets, blocks, loops of inextensible fabric, loops in inextensible cables, and whipple bars for engaging with the inextensible strands 1930A, 2430 of the tension member. The pulleys, threadable beads, rings, eyelets, blocks and whipple bars may be formed of any material capable of withstanding the tensions applied, including without limitation wood, metal, plastic and other suitable polymer materials. The elements may be formed by, for example without limitation, injection molding or three-dimensional printing technology.

Garment 1900, 2400 may be made of a garment material and each conduit may have a lower coefficient of friction with respect to strands 1930A, 2430 than the coefficient of friction of the garment material with respect to strands 1930A, 2430. Each of the conduits may comprise a tube of material different from the garment material. The three-dimensional spatial path of the tension member may be arranged so that tension in the tension member produces a compressive force proximate the joint.

Tensioner 1940A, 2440 may be engaged with tension evener 1942A, 2442 so as to provide a mechanical advantage in applying tension to the tension evener, as is shown for tensioner 1940A in FIGS. 16A, 16B, 16C, and 16D and FIGS. 17A, 17B, and 17C. The joint may be one of an ankle, elbow, wrist, shoulder, hip, neck, spine, and one or more vertebrae of the wearer.

One of the applications of the brace garment presented here is best described at the hand of FIG. 1, which shows the human left knee with the patella or knee cap and its various adhesions removed for the sake of clarity of the internal structure. Osteoarthritis is the painful condition in which the cartilage on tibia 120, being medial side of articular cartilage 140 and lateral side of articular cartilage 140, are worn down. It is generally the medial side of articular cartilage 140 that tends to exhibit the condition more frequently. One possible treatment of this condition is to reduce the pressure exerted by femur 110 on what remains (if any) of the medial side of articular cartilage 140. This may be achieved by the use of a brace garment of the type shown on the left leg in FIG. 14A, but with the apparatus instead applied to the outside or lateral side of the left knee joint, rather than to the medial side as depicted in FIG. 14A. The force applied to the lateral side of the left knee joint “unloads” the pressure on the medial side of articular cartilage 140 and reduces the wear and pain. Similar arrangements may be implemented for other joints in the human anatomy.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A wearable brace garment for stabilizing an articulating joint of a wearer, the garment configured for enveloping at least in part the joint, the garment comprising:
a tension member configured for being disposed along a three-dimensional spatial path about the joint, the tension member comprising a plurality of strands substantially inextensible along a longitudinal direction;
a tensioner disposed on the garment and arranged for adjusting a tension in the tension member; and
a tension evener disposed on the garment tension-wise in communication with the tensioner and with the strands of the tension member, the tension evener arranged for evening tension among the plurality of inextensible strands of the tension member.

2. The garment of claim 1, wherein the tension evener comprises a whippletree.

3. The garment of claim 2, wherein the whippletree is a multi-tier whippletree.

4. The garment of claim 2, wherein the whippletree comprises one or more pulleys disposed for engaging with the substantially inextensible strands of the tension member.

5. The garment of claim 4, wherein the one or more pulleys comprise of one of wood, metal, and a polymer, and wherein pulleys comprising polymer comprise one of injection-molded pulleys and three-dimensionally printed pulleys.

6. The garment of claim 2, wherein the whippletree comprises threadable members disposed for engaging with the substantially inextensible strands of the tension member.

7. The garment of claim 6, wherein the threadable members include one or more of beads, blocks, rings, eyelets and whipple bars disposed for engaging with the substantially inextensible strands of the tension member, and wherein the one or more of beads, blocks, rings, eyelets and whipple bars is made of one of wood, metal, and a polymer, wherein polymer threadable members are one of injection-molded and three-dimensionally printed.

8. The garment of claim 2, wherein the whippletree comprises a plurality of loops of one of substantially inextensible fabric and substantially inextensible cables disposed for engaging with the substantially inextensible strands of the tension member.

9. The garment of claim 1, further comprising a web portion comprising the strands laterally spread, each strand disposed in a corresponding strand conduit on or within the garment.

10. The garment of claim 9, wherein the garment is made of a garment material having a coefficient of friction and wherein each conduit has a lower coefficient of friction with respect to the strands than the coefficient of friction of the garment material with respect to the strands.

11. The garment of claim 9, wherein each conduit comprises a tube of material different from the garment material.

12. The garment of claim 9, wherein the tension evener comprises a whippletree.

13. The garment of claim 1, wherein the three-dimensional spatial path of the tension member is configured to extend around a limb articulating at the joint.

14. The garment of claim 1, wherein the three-dimensional spatial path of the tension member is configured and arranged so that tension in the tension member produces a compressive force proximate the joint.

15. The garment of claim 1, wherein the garment is configured to stabilize one of an ankle, elbow, wrist, shoulder, hip, neck, spine, and one or more vertebrae of the wearer.

16. A brace garment wearable by a wearer, the brace garment comprising a closed tensionable loop, a tensioner, and a tension evener, the tensioner disposed for applying a tension to the tensionable loop, the loop comprising a tension member arranged to substantially freely move along one or more conduits within or on the garment, wherein:
the tension member comprises a plurality of flexible and substantially longitudinally inextensible strands;
the garment comprises a web portion wherein the plurality of strands is laterally spread and each strand is disposed to move substantially freely within a corresponding strand conduit;
the tensionable loop is configured to be disposed on a joint of the wearer to create pressure on the joint of the wearer when the garment is worn by the wearer and the tensionable loop is tensioned; and
the tension evener is disposed to engage with the plurality of strands and is arranged to even among the plurality of strands the tension applied by the tensioner.

17. The garment of claim 16, wherein the brace garment is configured for the knee of a leg of the wearer and the web portion is configured to in part circle the leg at a distal region of a corresponding triceps surae of the wearer.

18. The garment of claim 17, wherein the tension member is configured to be disposed at least partially encircling a thigh of the leg.

19. The garment of claim 16, wherein the brace garment is configured to be disposed on a torso having a shoulder joint of the wearer and the web portion is configured to grip a portion of the torso opposing the shoulder joint of the wearer.

20. The garment of claim 19, wherein the tension member is configured to encircle the torso of the wearer.

21. The garment of claim 16, wherein the tension evener comprises a whippletree.

22. The garment of claim 21, wherein the whippletree comprises one of at least one pulley disposed for engaging with the substantially inextensible strands of the tension member, and threadable members disposed for engaging with the substantially inextensible strands of the tension member.

23. The garment of claim 22, wherein the threadable members include one or more of beads, blocks, rings, eyelets and whipple bars disposed for engaging with the substantially inextensible strands of the tension member.

24. The garment of claim 21, wherein the whippletree comprises a plurality of loops of one of substantially inextensible fabric and substantially inextensible cables disposed for engaging with the substantially inextensible strands of the tension member.
